(19) United States
(12) Patent Application Publication
(10) Pub. No.: US 2016/0367134 A1
(43) Pub. Date: Dec. 22, 2016

(54) WIDE FIELD OF VIEW OPTICAL COHERENCE TOMOGRAPHY IMAGING SYSTEM

(71) Applicant: Wei SU, Sunnyvale, CA (US)

(72) Inventor: Wei SU, Sunnyvale, CA (US)

(21) Appl. No.: 15/186,402

(22) Filed: Jun. 17, 2016

Related U.S. Application Data
(60) Provisional application No. 62/182,300, filed on Jun. 19, 2015.

(51) Int. Cl. A61B 3/10; A61B 3/14

(52) U.S. Cl. CPC A61B 3/102; A61B 3/14; G02B 27/141

(57) ABSTRACT

Disclosed herein is an optical coherence tomography (OCT) imaging system having a wide field of view and comprising a handheld imaging probe. The handheld imaging probe can comprise a first imaging module and an OCT imaging module. The first imaging module can have a first illumination path and a separated first imaging path. The first imaging module can comprise an optical window configured to be in contact with a sample. The OCT imaging module can comprise a scanning MEMS mirror and a beam splitting dichroic mirror. The OCT imaging system can comprise at least one polarization maintaining fiber to reduce motion effect to stabilize and increase the OCT image quality. The handheld imaging probe can further comprise one or more lenses achromatized for optical dispersion for the light beams within a wavelength range of an OCT light source and for a field of view of the OCT imaging module.

### CROSS REFERENCE TO RELATED APPLICATIONS

This application claims the benefit of U.S. Provisional Application No. 62/182,300, titled: “A WIDE FIELD OF VIEW EYE IMAGING APPARATUS WITH AN OPTICAL COHERENCE TOMOGRAPHY IMAGING SYSTEM”, filed Jun. 19, 2015, which is herein incorporated by reference in its entirety.

### INCORPORATION BY REFERENCE

The following U.S. patent applications are herein incorporated by reference in their entirety to the same extent as if each individual publication or patent application was specifically and individually indicated to be incorporated by reference: U.S. Pat. No. 9,155,466, titled “EYE IMAGING APPARATUS WITH A WIDE FIELD OF VIEW AND RELATED METHODS” and filed on Feb. 4, 2015, U.S. patent application Ser. No. 14/220,005, titled “EYE IMAGING APPARATUS AND SYSTEMS”, filed on Mar. 19, 2014, U.S. patent application Ser. No. 14/312,590, titled “MECHANICAL FEATURES OF AN EYE IMAGING APPARATUS”, filed on Jun. 23, 2014, U.S. patent application Ser. No. 15/007,101, titled: “A DISPOSABLE CAP FOR AN EYE IMAGING APPARATUS AND RELATED METHODS” and filed on Jan. 26, 2016, and U.S. Patent Application No. 62/141,209, titled “A WIRELESS IMAGING APPARATUS AND RELATED METHODS”, filed on Mar. 31, 2015.

All publications and patent applications mentioned in this specification are herein incorporated by reference in their entirety to the same extent as if each individual publication or patent application was specifically and individually indicated to be incorporated by reference.

### BACKGROUND

Various embodiments of the disclosure relate generally to an Optical Coherence Tomography (OCT) imaging system, particularly, to a wide field of view OCT imaging system.

Eyes are among the most valued human organs that play indispensable roles in life. Likewise, eye diseases and vision loss in general are serious problems. Moreover, eye diseases and vision problems among children, especially new-born babies, can have severe and far-reaching implications. For infants and small children, the visual centers in the brain are not fully mature. For the visual centers in the brain to develop properly, proper input from both eyes is desirable. Therefore good vision can be an important factor in the proper physical development and educational progress. Undetected eye problems in infants and others may result in irreversible loss of vision. Early detection and diagnosis provide the best opportunity for treatment and prevention of vision loss.

In eye examinations, eye imaging system has become increasingly important. Since retinal and optic nerve problems are among the leading causes in vision loss, eye imaging system capable of imaging a posterior segment of the eye can be particularly useful. Moreover, an eye imaging system with a wide field of view can offer the benefit of enabling evaluation of pathologies located on the periphery of the retina.

An OCT imaging system can provide the structure information of the eye, which is valuable for early diagnosis of eye diseases. In general, the OCT imaging system typically uses near-infrared light to obtain a cross sectional view of an eye, for example, a cross sectional view of the human retina.

For ophthalmic application, conventional OCT imaging systems provide a field of view (FOV) of about 20°×20°, measured from the nodal point of the eye. For many diseases such as diabetic retinopathy, vascular occlusions, a wider FOV is desirable. The technique of mosaicking has been used to achieve a larger FOV. However, a major drawback of mosaicking is the increased measurement time for a whole volume, including the time to realign the patient. Post-processing also becomes more complex with possible registration errors.

Moreover, most conventional OCT imaging systems have been non-contact type tabletop systems. In general, an OCT imaging system can include several subsystems such as an OCT console including a processing unit and a scanning subsystem. The scanning subsystem can provide the interface to a sample, for example, a patient, that is being imaged. The conventional OCT imaging systems typically have a fixed interface, where the patient is aligned with the location of the light coming from the OCT imaging system in order to obtain an OCT image. For example, the conventional interface has a chin rest for the patient and a mechanism for aligning the sample with the OCT imaging module. This system typically requires a mobile, upright, and cooperative patient in order to obtain usable OCT images.

However, the conventional non-contact OCT imaging systems require the cooperation of the patients, which is impractical for small children and infants since they can't follow the instruction of the operators. For adult patient who are bedridden or non-cooperative, the conventional non-contact OCT systems do not work, either. Furthermore, the conventional non-contact OCT systems will not be able to obtain OCT images of animal eyes as well.

Contact-type OCT imaging probes have been proposed recently. For example, a portable OCT imaging probe with a contact lens is proposed in U.S. Pat. No. 9,173,563. Portable probes may be useful in retinal and corneal imaging in infants or children, adults that are not cooperative or are bedridden, animals, etc. The contact probe makes physical contact between the probe and the patient, therefore alignment may be relatively simple since the tip of the probe can be visually placed on the sample in the desired location for imaging. A protective cover may be provided on the probe, for example, over the end of the probe, in order to reduce the likelihood of contamination of the probe and cross-contamination among different patients. However, the proposed contact-type OCT imaging probes, including the optical design in the above '563 patent, suffer from several problems. For example, the achievable FOV in the proposed contact-type OCT imaging probes is limited. In the '563 patent, the light source is disposed along the imaging path. The illumination path and the imaging path share the same optical path. Such design results in the reflected light from the cornea being much stronger than the reflected light from the retina. The problem is more severe in a wider angle, thus limiting the actual achievable FOV of the proposed contact-type OCT imaging probes. For another example, the proposed contact-type OCT imaging probes further have sensitivity to the motion of the fibers connecting the portable probes to the console, and have distortion caused by the motion of the fibers during the operation. The motion effect of the fibers may significantly decrease the stabilization and the optical quality of the OCT images.

There is a pressing need to develop a contact-type OCT imaging system with a wide FOV, which is suitable for infants, small children, bedridden patients, animals, etc.

### SUMMARY OF THE DISCLOSURE

The present disclosure relates to a wide field of view optical coherence tomography (OCT) imaging system. Various embodiments of the present disclosure describe an optical coherence tomography (OCT) imaging system having a wide field of view. The OCT imaging system can comprise a handheld imaging probe. The handheld imaging probe can comprise a probe housing with a distal end, a first imaging module (for example, a color imaging module) and an OCT imaging module, which is a second imaging module. The first imaging module can have a first illumination path and a first imaging path. The first imaging module can comprise a first light source disposed inside the housing, where the first light source can have a first wavelength range. The first imaging module can comprise an optical window disposed at the distal end and configured to be in contact with a cornea of an eye. The optical window can have a concave front surface with a radius of curvature of from about 3 mm to about 18 mm. The optical window can be in contact of the cornea directly, or indirectly through a disposable cap. The index matching gel can be applied between the optical window and the cornea. The optical window is configured to match a radius of curvature of the cornea of the eye. The first imaging module can comprise a light conditioning element having multiple segments and positioned behind the peripheral portion of the optical window, the light conditioning element configured to directionally control a first light beam of the first light source to illuminate the eye through the first illumination path. The first imaging module can comprise a first focusing lens to adjust a first focus of the first imaging module, and an image sensor configured to receive a first image of the eye through the first imaging path. The first imaging path is separated from the first illumination path. The first imaging path does not overlap with the first illumination path. The light conditioning element is configured to direct substantially all the light exiting the light conditioning element outside an entrance pupil of the first imaging system. Further details of the first imaging module are disclosed in U.S. Pat. No. 9,155,466, which is herein incorporated by reference in its entirety.

The handheld imaging probe can comprise an OCT imaging module, which can have a second illumination path and a second imaging path. The OCT imaging module can comprise a scanning MEMS mirror configured to scan a first portion of a second light beam from a second light source. The second light source can be an OCT light source, such as a broadband light source. In some embodiments, the second light source is a swept source laser, which can provide a fast wavelength sweeping speed. The scanning MEMS mirror is disposed outside the first illumination path and the first imaging path. The OCT imaging module can comprise a beam splitting dichroic mirror disposed in the first imaging path and configured to transmit the first light beam and reflect the first portion of the second light beam, and a second focusing lens to adjust a second focus of the second imaging module.

The OCT imaging module can have a wide field of view. The field of view measured from the nodal point of the eye, may in certain embodiments be at least 60 degrees×60 degrees and up to 180 degree×180 degrees in a single volume acquisition. In some embodiments, the field of view is at least 120 degrees×120 degrees but no more than 180 degrees×180 degrees in a single volume acquisition. In some embodiments, the field of view is at least 130 degrees×130 degrees but no more than 180 degrees×180 degrees in a single volume acquisition.

In general, the OCT imaging system can comprise the handheld imaging probe and a console. In general, the imaging probe can comprise a first imaging module, for example, an optical color imaging module or a full filed imaging module, and an OCT imaging module. In general, the console can comprise an OCT engine and a scanning mirror controller. The OCT engine can comprise an OCT interferometer, an OCT light source (for example, a broadband light source with wavelength from 800 nm to 1100 nm), a light detector, a data acquisition system, a power supply, a processor, and a display. The OCT engine can be Time-domain OCT engine (TD-OCT), Fourier-domain OCT engine (FD-OCT), or Swept source Fourier-domain OCT engine (SS-FD-OCT).

Disclosed herein is a wide field of view OCT imaging system. In general, the OCT imaging system can comprise a handheld imaging probe. The handheld imaging probe can comprise a probe housing with a distal end, a first imaging module and an OCT imaging module. The first imaging module can be disposed on a main portion of the probe housing. The OCT imaging module can be disposed on a side portion of the probe housing.

The first imaging module can comprise a first illumination path and a first imaging path, where the first imaging path is separated from the first illumination path. The first illumination path comprises a first light source disposed inside the probe housing and a light conditioning element having multiple segments. The light conditioning element is positioned behind a peripheral portion of an optical window. The light conditioning element is configured to receive a first light beam from the first light source and directionally control the first light beam to an eye. The first imaging path can comprise an optical window disposed at the distal end of the probe housing and configured to be in contact with a cornea of the eye. The optical window can have a concave front surface. The first imaging path can comprise one or more lenses, which include a first focusing lens to adjust a focus of the first imaging module. The first imaging path can further comprise an image sensor configured to receive a first image of the eye.

The OCT imaging module can comprise a second illumination path and a second imaging path. The second illumination path and the second imaging path can comprise a scanning MEMS mirror and a beam splitting dichroic mirror. The scanning MEMS mirror is configured to scan a first portion of an OCT light beam from an OCT light source. The scanning MEMS mirror can be disposed outside the first illumination path and the first imaging path. The beam splitting dichroic mirror can be disposed in the first imaging path and configured to transmit the first light beam and reflect the first portion of the OCT light beam. The second illumination path and the second imaging path can further comprise a second focusing lens to adjust a focus of the OCT imaging module.

In some embodiments the scanning MEMS mirror is positioned in an optical conjugate plane of an entrance pupil of the first imaging system. In some embodiments a real image of an aperture of the OCT imaging module is positioned near an anterior surface of the crystalline lens of the eye when a posterior segment of the eye is imaged.

In some embodiments the OCT imaging system can further comprise an imaging lens and a first relay lens disposed in the first imaging path and optically aligned with the optical window, where the beam splitting dichroic mirror is disposed behind the first relay lens. The second imaging path and the first imaging path share optical components only until the first relay lens.

In some embodiments a field of view of the OCT imaging module is at least 120 degrees×120 degrees but no more than 180 degrees×180 degrees in a single volume acquisition. In some embodiments the first light source has a first wavelength range between 450 nm to 700 nm, inclusive. In some embodiments the first light source has a first wavelength range from 700 nm to 840 nm in the near infrared light range.

In some embodiments the optical window and the OCT imaging module are disposed on a removable front imaging module of the handheld imaging probe, wherein the removable front imaging module are configured to be repeatedly detached from, and re-attached to, the handheld imaging probe without using tools.

In some embodiments the optical lenses in the first imaging path including the first focusing lens is achromatized for optical dispersion for the first light beam within a wavelength range of the first light source and for a field of view of the first imaging module. In some embodiments the optical lenses in the second imaging path including the second focusing lens is achromatized for optical dispersion for the OCT light beam within a wavelength range of the OCT light source and for a field of view of the OCT imaging module.

In some embodiments the OCT imaging system can further comprise a console, wherein the console comprises the OCT light source, an interferometer, and a first optical fiber, wherein the first optical fiber is connected to the console and the handheld imaging probe and configured to couple the first portion of the OCT light beam from the OCT light source to the handheld imaging probe to form a sample arm of the interferometer. In some embodiments the OCT light source is a swept-source laser. In some embodiments the console further comprises a second optical fiber configured to couple the second portion of the OCT light beam to a reference arm of the interferometer, the reference arm disposed in the console. In some other embodiments the handheld imaging probe further comprises a reference arm of the OCT interferometer with its light beam transmitted in the same first optical fiber for the sample arm, wherein the second portion of the OCT light beam is coupled to the reference arm by the first optical fiber.

In some embodiments the OCT imaging system can further comprise a processor configured to process data from the interferometer and to generate an OCT image of the eye from the OCT imaging module. In some embodiments the handheld imaging probe can further comprise a wireless transmitter, a wireless receiver and a display, the display configured to present the first image and the OCT image simultaneously.

In some embodiments the first optical fiber is configured to maintain a polarization of the OCT light beam to reduce motion effect of the first optical fiber to stabilize and increase the OCT image quality. In some embodiments the first optical fiber comprises a plurality of turns to remove the residual light beam in one of axes of the optical fiber such that the light beam transmitting in the first with only one linear polarization.

In some embodiments the OCT imaging system can further comprise a scanning MEMS mirror controller disposed in the console and an electrical cable connecting the console to the handheld imaging probe, wherein the scanning mirror controller and the scanning MEMS mirror are synchronized through the electrical cable.

In some embodiments the OCT imaging system can further comprise a scanning MEMS mirror controller disposed in the console, an electrical-optical converter disposed in the console and configured to convert an electrical signal into an optical signal, an optical-electrical converter disposed in the handheld imaging probe and configured to convert the optical signal back into the electrical signal, and a third optical fiber connected to the console to the handheld imaging probe, the third optical fiber is configured to transmit the optical signal, wherein the scanning mirror controller, the scanning mirror driver and the scanning MEMS mirror are synchronized through the third optical fiber.

In some embodiments the OCT imaging system can further comprise a pair of wireless transponders, one wireless transponder disposed in the console and the other wireless transponder disposed in the handheld imaging probe, wherein the scanning mirror controller and the scanning MEMS mirror are synchronized wirelessly via the wireless transponders.

In some embodiments the OCT imaging system can further comprise a third light source disposed in the console and a beam combiner disposed in the console, the beam combiner is configured to couple both the OCT light source and the third light source to the handheld imaging probe through the first optical fiber, the third light source having a third light beam, the third light beam having a third illumination path and a third imaging path, wherein the third illumination path is along the second illumination path of the OCT imaging module and the third imaging path is along the first imaging path of the first imaging module, wherein the beam splitting dichroic mirror is configured to partially reflect and partially transmit the third light beam. In some embodiments a track of the third light beam is configured to provide registry of an imaging location of the OCT light beam and provide a feedback to control the second focusing lens, wherein a first adjustment of the first focusing lens and a second adjustment of the second focusing lens are synchronized through the feedback.

One aspect of the disclosure is a wide field of view optical coherence tomography (OCT) imaging system. The wide field of view OCT system can comprise a handheld imaging probe. The handheld imaging probe can have an OCT imaging module. The handheld imaging probe can comprise a probe housing with a distal end and an optical window disposed at the distal end. The optical window can have a concave front surface and is configured to be in contact with a cornea of an eye. The handheld imaging probe can further comprise a scanning MEMS mirror configured to scan a first portion of a light beam from a light source. The handheld imaging probe can further comprise one or more lenses in an imaging path. The wide field of view OCT system can comprise a console. The console can comprise the light source, an interferometer, a processor, a scanning MEMS mirror controller and a datalink. The interferometer can comprise at least one polarization maintaining fiber, the at least one polarization maintaining fiber can be configured to couple a light beam from the light source to the handheld imaging probe and reduce motion effect to stabilize and increase a quality of an OCT image. The processor can be configured to process data from the interferometer and to generate the OCT image of the eye from the OCT imaging module. The data link is connected to the console and the handheld imaging probe and configured to synchronize the scanning MEMS mirror with the light source and the scanning MEMS mirror controller.

In some embodiments a field-of view of the OCT imaging module is at least 120 degrees×120 degrees but no more than 180 degrees×180 degrees in a single volume acquisition.

In some embodiments the one or more lenses are achromatized for optical dispersion for the light beams within a wavelength range of the light source. In some embodiments the one or more lenses are achromatized for optical dispersion for a field of view of the OCT imaging module. In some embodiments the light source is a swept-source laser.

In some embodiments the at least one polarization maintaining fiber comprises a plurality of turns to remove the residual OCT light beam in one of axes of the polarization maintaining fiber such that the light beam transmitting with only one linear polarization.

In some embodiments the data link is an electrical cable. In some embodiments the data link is a second optical fiber cable. In some embodiments the data link is wireless.

In some embodiments the OCT imaging system can further comprise a wireless transmitter, a wireless receiver and a display, the display configured to present the OCT image.

Various embodiments disclosed herein comprise a wide field of view optical coherence tomography (OCT) imaging system. The wide field of view OCT system can comprise a handheld imaging probe. The handheld imaging probe can have an OCT imaging module. The handheld imaging probe can comprise a probe housing with a distal end and an optical window disposed at the distal end. The optical window can have a concave front surface and is configured to be in contact with a cornea of an eye. The handheld imaging probe can further comprise a scanning MEMS mirror configured to scan a first portion of a light beam from a light source. The handheld imaging probe can further comprise one or more lenses in an imaging path, where the one or more lenses are achromatized for optical dispersion for the light beams within a wavelength range of the light source and for a field of view of the OCT imaging module. The OCT system can comprise a console. The console can comprise the light source, an interferometer, a processor, a scanning MEMS mirror controller and a datalink. The processor can be configured to process data from the interferometer and to generate the OCT image of the eye from the OCT imaging module. The data link is connected to the console and the handheld imaging probe and configured to synchronize the scanning MEMS mirror with the light source and the scanning MEMS mirror controller.

In some embodiments a field-of view of the OCT imaging module is at least 120 degrees×120 degrees but no more than 180 degrees×180 degrees in a single volume acquisition. In some embodiments the light source is a swept-source laser. In some embodiments the data link is an electrical cable. In some embodiments the data link is another optical fiber cable. In some embodiments the data link is wireless. In some embodiments the OCT imaging system can further comprise a wireless transmitter, a wireless receiver and a display, the display configured to present the OCT image.

Various embodiments disclosed herein describe a method of obtaining a wide field of view optical coherence tomography (OCT) image. The method can comprise holding a handheld imaging probe in a hand, placing an optical window disposed at a distal end of the handheld imaging probe in contact with a cornea of an eye, illuminating the eye with a first light beam transmitted from a first light source through a light conditioning element. The light conditioning element is positioned behind a peripheral portion of the optical window along a first illumination path. The method further comprise directional controlling the first light beam with the light conditioning element and obtaining a first image of the eye through a first imaging path, the first imaging path is separated from the first illumination path. The method further comprise scanning an OCT light beam from an OCT light source with a scanning MEMS mirror, the scanning MEMS mirror is disposed outside the first illumination path and the first imaging path. The method further comprise reflecting the first portion of the OCT light beam off of a beam splitting dichroic mirror to the eye, and obtaining the OCT image of the eye.

In some embodiments obtaining a first image of the eye comprises obtaining a first image of the eye with one or more lenses achromatized for optical dispersion for the first light beam within a wavelength range of the first light source and for a field of view of the first image.

The method can further comprise obtaining the OCT image of the eye comprises obtaining the OCT image of the eye with one or more lenses achromatized for optical dispersion for the OCT light beam within a wavelength range of the OCT light source and for a field of view of the OCT image. The method can further comprise using a swept-source laser as the OCT light source. The method can further comprise presenting the first image and the OCT image simultaneously on a display on the handheld imaging probe and console simultaneously.

The method can further comprise coupling the OCT light beam from the OCT light source to the handheld imaging probe with a polarization maintaining optical fiber to reduce motion effect to stabilize and increase the OCT image quality.

The method can further comprise providing registry of an imaging location of the OCT light beam with a third light beam from a third light source, the third light beam having a third illumination path along an illumination path of the OCT imaging module and a third imaging path along the first imaging path of the first imaging module. The method can further comprise synchronizing a first focus adjustment of the first image and a second focus adjustment of the OCT image with a feedback from a track of the third light beam.

One aspect of the disclosure is a method of obtaining a wide field of view optical coherence tomography (OCT) image. The method can comprise holding a handheld imaging probe in a hand, and placing an optical window disposed at a distal end of the imaging probe in contact with a cornea of an eye. The method can further comprise illuminating the eye with an OCT light beam from an OCT light source. The OCT light source can be coupled to the handheld imaging probe by a polarization maintaining fiber to reduce motion effect and to increase OCT image quality. The method can further comprise scanning a first portion of the OCT light beam by using a scanning MEMS mirror, and obtaining the OCT image of the eye.

In some embodiments obtaining the OCT image of the eye comprises obtaining the OCT image of the eye with one or more lenses achromatized for optical dispersion for the OCT light beam within a wavelength range of the OCT light source and for a field of view of the OCT image. In some embodiments the method can further comprise using a swept-source laser as the OCT light source. The method can further comprise presenting the OCT image on a display on the handheld imaging probe.

The method can further comprise illuminating the eye with an OCT light beam comprises illuminating the eye with an OCT light beam by coupling the OCT light beam from the OCT light source to the handheld imaging probe with a polarization maintaining optical fiber with a plurality of turns to remove the residual OCT light beam in one of axes of the polarization maintaining optical fiber.

Various embodiments of the disclosure comprise a method of obtaining a wide field of view optical coherence tomography (OCT) image. The method can comprise holding a handheld imaging probe in a hand, and placing an optical window disposed at a distal end of the imaging probe in contact with a cornea of an eye. The method can further comprise illuminating the eye with an OCT light beam from an OCT light source optically coupled to the handheld imaging probe. The method can further comprise scanning a first portion of the OCT light beam by using a scanning MEMS mirror. The method can further comprise obtaining the OCT image through one or more lenses in an imaging path, the one or more lenses are achromatized for optical dispersion for the light beams within a wavelength range of the OCT light source and for a field of view of an OCT imaging module disposed in the handheld imaging probe to increase the OCT image quality. The method can further comprise using a swept-source laser as the OCT light source. The method can further comprise presenting the OCT image on a display on the handheld imaging probe.

In general, disclosed herein is a wide field of view OCT imaging system for a variety of applications, not being limited to ophthalmic application. For example, the OCT imaging system can be used to image an ear, a nose or other body parts of human, or a variety of other samples. The OCT imaging system can comprise a handheld imaging probe and a console. The handheld imaging probe can comprise a probe housing with a distal end, a first imaging module and an OCT imaging module. The first imaging module can be disposed on a main portion of the probe housing. The OCT imaging module can be disposed on a side portion of the probe housing.

The first imaging module can comprise a first illumination path and a first imaging path, where the first imaging path is separated from the first illumination path. The first illumination path comprises a first light source disposed inside the probe housing and a light conditioning element having multiple segments. The light conditioning element is positioned behind a peripheral portion of an optical window. The light conditioning element is configured to receive a first light beam from the first light source and directionally control the first light beam to a sample. The first imaging path can comprise an optical window disposed at the distal end of the probe housing and configured to be in contact with the sample. The optical window can have a front surface mating a shape of the sample to be viewed. The first imaging path can comprise one or more lenses, which include a first focusing lens to adjust a focus of the first imaging module. The first imaging path can further comprise an image sensor configured to receive a first image of the sample. The OCT imaging module can comprise a second illumination path and a second imaging path. The second illumination path and the second imaging path can comprise a scanning MEMS mirror and a beam splitting dichroic mirror. The scanning MEMS mirror is configured to scan a first portion of an OCT light beam from an OCT light source. The scanning MEMS mirror can be disposed outside the first illumination path and the first imaging path. The beam splitting dichroic mirror can be disposed in the first imaging path and configured to transmit the first light beam and reflect the first portion of the OCT light beam. The second illumination path and the second imaging path can further comprise a second focusing lens to adjust a focus of the OCT imaging module.

One aspect of the disclosure is a wide field of view optical coherence tomography (OCT) imaging system. The wide field of view OCT system can comprise a handheld imaging probe. The handheld imaging probe can have an OCT imaging module. The handheld imaging probe can comprise a probe housing with a distal end and an optical window disposed at the distal end. The optical window can have a front surface matching a shape of a sample and is configured to be in contact with the sample. The handheld imaging probe can further comprise a scanning MEMS mirror configured to scan a first portion of a light beam from a light source. The handheld imaging probe can further comprise one or more lenses in an imaging path. The wide field of view OCT system can comprise a console. The console can comprise the light source, an interferometer, a processor, a scanning MEMS mirror controller and a datalink. The interferometer can comprise at least one polarization maintaining fiber, the at least one polarization maintaining fiber can be configured to couple a light beam from the light source to the handheld imaging probe and reduce motion effect to stabilize and increase a quality of an OCT image. The processor can be configured to process data from the interferometer and to generate the OCT image of the sample from the OCT imaging module. The data link is connected to the console and the handheld imaging probe and configured to synchronize the scanning MEMS mirror with the light source and the scanning MEMS mirror controller.

Another aspect of the disclosure is a wide field of view optical coherence tomography (OCT) imaging system. The wide field of view OCT system can comprise a handheld imaging probe. The handheld imaging probe can have an OCT imaging module. The handheld imaging probe can comprise a probe housing with a distal end and an optical window disposed at the distal end. The optical window can have a front surface matching a shape of a sample and is configured to be in contact with the sample. The handheld imaging probe can further comprise a scanning MEMS mirror configured to scan a first portion of a light beam from a light source. The handheld imaging probe can further comprise one or more lenses in an imaging path, where the one or more lenses are achromatized for optical dispersion for the light beams within a wavelength range of the light source and for a field of view of the OCT imaging module. The OCT system can comprise a console. The console can comprise the light source, an interferometer, a processor, a scanning MEMS mirror controller and a datalink. The processor can be configured to process data from the interferometer and to generate the OCT image of the sample from the OCT imaging module. The data link is connected to the console and the handheld imaging probe and configured to synchronize the scanning MEMS mirror with the light source and the scanning MEMS mirror controller.

One aspect of the disclosure is a method of obtaining a wide field of view optical coherence tomography (OCT) image. The method can comprise holding a handheld imaging probe in a hand, placing an optical window disposed at a distal end of the handheld imaging probe in contact with a sample, illuminating the sample with a first light beam transmitted from a first light source through a light conditioning element. The light conditioning element is positioned behind a peripheral portion of the optical window along a first illumination path. The method further comprise directional controlling the first light beam with the light conditioning element and obtaining a first image of the sample through a first imaging path, the first imaging path is separated from the first illumination path. The method further comprise scanning an OCT light beam from an OCT light source with a scanning MEMS mirror, the scanning MEMS mirror is disposed outside the first illumination path and the first imaging path. The method further comprise reflecting the first portion of the OCT light beam off of a beam splitting dichroic mirror to the eye, and obtaining the OCT image of the sample.

One aspect of the disclosure is a method of obtaining a wide field of view optical coherence tomography (OCT) image. The method can comprise holding a handheld imaging probe in a hand, and placing an optical window disposed at a distal end of the imaging probe in contact with a sample. The method can further comprise illuminating the sample with an OCT light beam from an OCT light source. The OCT light source can be coupled to the handheld imaging probe by a polarization maintaining fiber to reduce motion effect and to increase OCT image quality. The method can further comprise scanning a first portion of the OCT light beam by using a scanning MEMS mirror, and obtaining the OCT image of the sample.

Another aspect of the disclosure is a method of obtaining a wide field of view optical coherence tomography (OCT) image. The method can comprise holding a handheld imaging probe in a hand, and placing an optical window disposed at a distal end of the imaging probe in contact with a sample. The method can further comprise illuminating the sample with an OCT light beam from an OCT light source optically coupled to the handheld imaging probe. The method can further comprise scanning a first portion of the OCT light beam by using a scanning MEMS mirror. The method can further comprise obtaining the OCT image through one or more lenses in an imaging path, the one or more lenses are achromatized for optical dispersion for the light beams within a wavelength range of the OCT light source and for a field of view of an OCT imaging module disposed in the handheld imaging probe to increase the OCT image quality. The method can further comprise using a swept-source laser as the OCT light source. The method can further comprise presenting the OCT image on a display on the handheld imaging probe.

### BRIEF DESCRIPTION OF THE DRAWINGS

FIG. 1 is a side view of a wide field of view OCT imaging system comprising a handheld imaging probe and a console.

FIG. 2A is a schematic of an example optical design of the wide field of view OCT imaging system in FIG. 1, where a posterior segment of an eye is imaged, according to one embodiment of the disclosure.

FIG. 2B is a side view of a distal end of a handheld imaging probe of the wide field of view OCT imaging system in FIG. 2A.

FIG. 2C is a perspective view of a light conditioning element in a first imaging module of the wide field of view OCT imaging system in FIG. 2A.

FIG. 3 is a schematic of another example optical design of the wide field of view OCT imaging system in FIG. 1, where a posterior segment of an eye is imaged, according to another embodiment of the disclosure.

FIG. 4 is a schematic of an example fiber optical interferometer of the wide field of view OCT imaging system in FIG. 1, according to one embodiment of the disclosure.

FIG. 5A is a schematic of an electrical cable as a date link to connect the console and the handheld imaging probe of the wide field of view OCT imaging system in FIG. 1, according to one embodiment of the disclosure.

FIG. 5B is a schematic of a second optical fiber cable as the date link to connect the console and the handheld imaging probe of the wide field of view OCT imaging system in FIG. 1, according to another embodiment of the disclosure.

FIG. 5C schematically illustrates the details of the synchronization between the broadband light source, the data acquisition system and the scanning MEMS mirror through the optical fiber cable.

FIG. 5D is a schematic that illustrates an electrical isolator is used to isolate the console and the handheld imaging probe of the wide field of view OCT imaging system in FIG. 1, according to yet another embodiment of the disclosure.

FIG. 6 is a schematic of a wireless data link to connect the console and the handheld imaging probe of a wide field of view OCT imaging system, according to an alternative embodiment of the disclosure.

FIG. 7 schematically illustrates the details of the synchronization between the broadband light source, the data acquisition system and the scanning MEMS mirror wirelessly.

FIG. 8A a side view of a handheld OCT imaging probe where an OCT imaging module is an integral part of a wide field of view OCT imaging system according to one embodiment of the disclosure.

FIG. 8B a side view of a handheld OCT imaging probe where an OCT imaging module can be detached from and repeatedly reattached to the main portion of the imaging probe of a wide field of view OCT imaging system according to another embodiment of the disclosure.

FIG. 9 is a schematic of a wide field of view OCT imaging system, where an anterior segment of an eye is imaged, according to one embodiment of the disclosure.

FIG. 10 is a schematic of an example optical design of a wide field of view OCT imaging system, where a reference arm of an OCT interferometer is integrated into a handheld OCT imaging probe according to another embodiment of the disclosure.

### DETAILED DESCRIPTION

The present disclosure now will be described in detail with reference to the accompanying figures. This disclosure may be embodied in many different forms and should not be construed as limited to the exemplary embodiments discussed herein.

Various embodiments of the present disclosure describe an optical coherence tomography (OCT) imaging system having a wide field of view. The OCT imaging system can comprise a handheld imaging probe. The handheld imaging probe can comprise a probe housing with a distal end, a first imaging module (for example, a color imaging module) and an OCT imaging module, which is a second imaging module. The first imaging module can have a first illumination path and a first imaging path. The first imaging module can comprise a first light source disposed inside the housing, where the first light source can have a first wavelength range. The first imaging module can comprise an optical window disposed at the distal end and configured to be in contact with a cornea of an eye. The optical window can have a concave front surface with a radius of curvature of from about 3 mm to about 18 mm. The optical window can be in contact of the cornea directly, or indirectly through a disposable cap. The index matching gel can be applied between the optical window and the cornea. The optical window is configured to match a radius of curvature of the cornea of the eye. The first imaging module can comprise a light conditioning element having multiple segments and positioned behind the peripheral portion of the optical window, the light conditioning element configured to directionally control a first light beam of the first light source to illuminate the eye through the first illumination path. The first imaging module can comprise a first focusing lens to adjust a first focus of the first imaging module, and an image sensor configured to receive a first image of the eye through the first imaging path. The first imaging path is separated from the first illumination path. The first imaging path does not overlap with the first illumination path. The light conditioning element is configured to direct substantially all the light exiting the light conditioning element outside an entrance pupil of the first imaging system. Further details of the first imaging module are disclosed in U.S. Pat. No. 9,155,466, which is herein incorporated by reference in its entirety.

The handheld imaging probe can comprise an OCT imaging module, which can have a second illumination path and a second imaging path. The OCT imaging module can comprise a scanning MEMS mirror configured to scan a first portion of a second light beam from a second light source. The second light source can be an OCT light source, such as a broadband light source. In some embodiments, the second light source is a swept source laser, which can provide a fast scanning speed. The scanning MEMS mirror is disposed outside the first illumination path and the first imaging path. The OCT imaging module can comprise a beam splitting dichroic mirror disposed in the first imaging path and configured to transmit the first light beam and reflect the first portion of the second light beam, and a second focusing lens to adjust a second focus of the second imaging module.

The OCT imaging module can have a wide field of view. The field of view measured from the nodal point of the eye, may in certain embodiments be at least 60 degrees×60 degrees and up to 180 degree×180 degrees in a single volume acquisition. In some embodiments, the field of view is at least 120 degrees×120 degrees but no more than 180 degrees×180 degrees in a single volume acquisition. In some embodiments, the field of view is at least 130 degrees×130 degrees but no more than 180 degrees×180 degrees in a single volume acquisition.

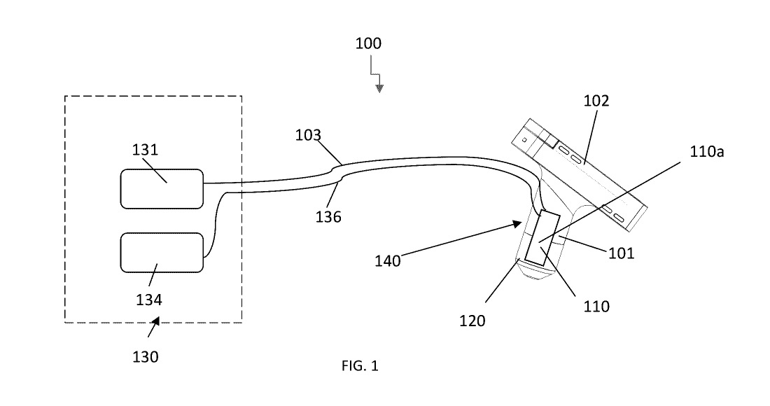

FIG. 1 is a side view of an OCT imaging system 100 with a wide field of view. The OCT imaging system 100 can comprise a handheld imaging probe 140 and a console 130. In general, the imaging probe 140 can comprise a first imaging module 120, for example, an optical color imaging module or a full filed imaging module, and an OCT imaging module 110. In general, the console 130 can comprise an OCT engine 131 and a scanning mirror controller 134. The OCT engine 131 can comprise an OCT interferometer, an OCT light source (for example, a broadband light source with wavelength from 800 nm to 1100 nm), a light detector, a data acquisition system, a power supply, a processor, and a display. The OCT engine 131 can be Time-domain OCT engine (TD-OCT), Fourier-domain OCT engine (FD-OCT), or Swept source Fourier-domain OCT engine (SS-FD-OCT).

The handheld imaging probe 140 can be compact, light-weight and portable. The imaging probe 140 can comprise the first imaging module (for example, a color imaging module) 120 and the OCT imaging module 110. In general, the imaging probe 140 houses not only the OCT Imaging Module, but also comes with other imaging modalities, for example, color imaging, fluorescine angiography imaging, auto-fluorescence imaging, etc. The imaging probe 140 can perform real-time OCT imaging with other imaging modality at same time, for example, the optical color imaging. In some embodiments, the optical color imaging module can be configured to provide registry of imaging locations for the OCT sectional views, which will be discussed in details below.

As shown in FIG. 1, an optical fiber 103 can be configured to transmit light from the OCT light source in the OCT engine 131 to the imaging probe 140. The OCT engine 131 can be linked, optically with the OCT Imaging Module 110 in the imaging probe 140 by an optical fiber, which supplies the light from the OCT light source to the OCT Imaging Module 110 and at same time, receive reflected light of the eye from the OCT Imaging Module 110 for further analysis. The OCT Imaging Module 110 scans the light beam from the OCT light source, by a scanning mirror (rotating one dimensionally or two dimensionally) disposed inside the handheld imaging probe 140, to the different parts of the eye (a posterior segment of the eye in the posterior imaging mode and an anterior segment of the eye in the anterior imaging mode) and receives the light reflected, or scattered, from the eye. For example, the scanning mirror can be a scanning MEMS mirror in some embodiments.

The handheld imaging probe 140 may be compact and in various embodiments, the imaging probe 140 may have a size less than 250 mm along the longest dimension thereof. For example, in some embodiments the imaging probe 140 may be between 250 mm and 200 mm, 150 mm, or 100 mm along the longest dimension. In some embodiments, the imaging probe 140 may weigh less than 1 kg. For example, the imaging probe 140 may weigh between 1 kg and 0.5 kg, or 0.3 kg, or 0.2 kg in some embodiments.

The OCT imaging system 100 including the imaging probe 140, the console 130 and the fiber cable 103 may be carried by the users in a small carrying case with a handle, for example, that is less than 600 mm×400 mm×300 mm and weigh less than 15 kg or in another convenient manner due to its compactness. In some embodiments, for example, the carrying case is between (600 mm and 300 mm)×(400 mm and 200 mm)×(300 and 150 mm). Also, the carrying case weighs between 15 kg and 10 kg or 5 kg, in some embodiments. Sizes outside these ranges for the OCT imaging system 100 and the carrying case are also possible. Various embodiments may be easily operated by the operators with little training.

The imaging probe 140 of the imaging apparatus 100 may have a main portion 101, for example, a handle portion constructed to be in a cylindrical shape to allow easy grabbing by one hand and usable as a handle. The main portion is extending longitudinally from a distal end of the probe housing. The main portion may be as disclosed in detail in U.S. patent application Ser. No. 14/220,005, which is herein incorporated by reference in its entirety. The users may precisely adjust the position/angle of the imaging probe 140 with one hand, freeing another hand to work on other tasks, for example, opening the eyelids of the patient with the fingers. The first imaging module (for example, the color imaging module) can be disposed inside the handle portion 101.

The OCT imaging module 110 can be disposed on a side portion 110a, which is attached to the main portion 101. In some embodiments, the imaging probe 140 can comprise a handgrip with a bump and the handgrip can be the side portion. The handgrip portion with a bump may be as disclosed in U.S. patent application Ser. No. 14/312,590, which is herein incorporated by reference in its entirety. The part of OCT imaging module 110 can be positioned inside the side portion 110a. For example, the side portion 110a can be shaped to fit with a palm of an operator holding the Imaging Probe 140. In some embodiments, the diameter of the cylindrical handgrip can be 20 mm to 80 mm, for example, the diameter of the cylindrical handgrip can be 30 mm to 50 mm. In some embodiments, the length of the cylindrical handgrip can be 60 mm to 300 mm, for example, the length of the cylindrical handgrip can be from 100 mm to 200 mm. The housing of the OCT imaging module 110, for example, the side portion 110a, can have a width of from 10 mm to 50 mm, a height of from 10-80 mm and a length of from 20 mm to 100 mm. For example, the side portion 110a, which can be the housing of the OCT imaging module 110, can be about 20-30 mm wide, 30-50 mm high and 40-60 mm long. Values outside the above ranges are also possible.

Captured images may be transferred to other computing devices or internet based devices, like storage units, through wired or wireless communication systems. The imaging probe 140 can further comprise a processor to control the first imaging module and the OCT imaging module. The imaging probe 140 can further comprise a wireless transmitter and a wireless receiver to communicate with the console 130 or other computing devices or internet based devices wirelessly. The imaging probe 140 can comprise a display 102 with a user input interface such as a touch screen monitor mounted at the top of the handle portion 101. The display 102 can be configured to display a first image from the first imaging system and a second cross sectional image from the OCT imaging module 110 simultaneously. In some embodiments, the imaging probe 140 is powered by a battery. Also in various embodiments, live images may be displayed on the touch screen monitor or a larger display monitor that receives data from this imaging probe 140 in real time.

The OCT imaging system 100 may be used as a disease screening or medical diagnosis device for the ophthalmic applications. It may be used in remote rural areas where traveling to the eye care facilities is not convenient. The OCT imaging system 100 may also be used as a portable medical imaging device to view other parts of the body (for example, ear or skin, etc.) for other medical needs such as ENT, dermatology, etc. Furthermore, the OCT imaging system 100 may have applications in areas other than medical applications, for example, for security screening applications where the images from the posterior/anterior segment of the eye may be used for the personal identification purpose. The OCT imaging system 100 may also be used to view a variety of samples including animals. The OCT imaging system 100 may also have other industry applications.

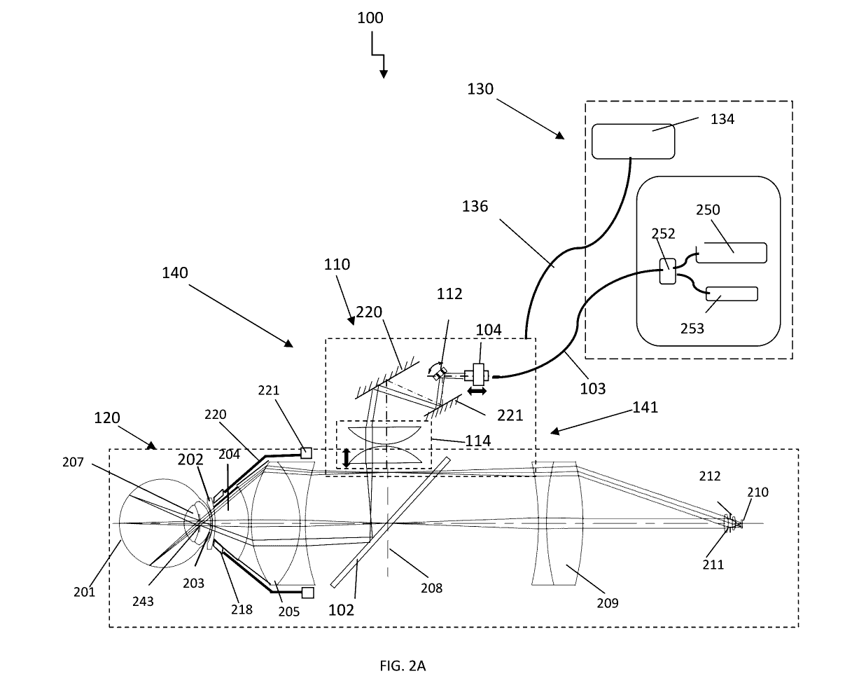

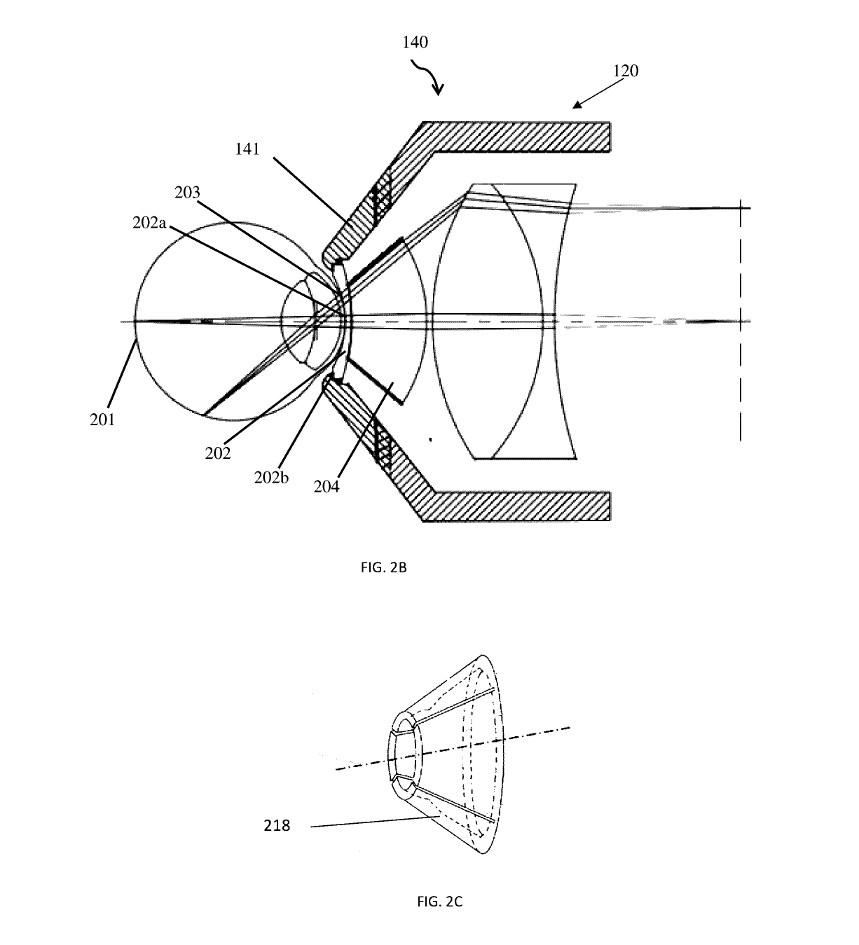

FIG. 2A schematically illustrates an example optical design of a wide field of view OCT imaging system 100 where a posterior segment 201 of an eye is imaged by the OCT imaging system 100 according to one embodiment of the disclosure. FIG. 2B is a side view of a distal end of the handheld imaging probe 140 of the OCT imaging system 100. Referring to FIG. 2A and FIG. 2B, the OCT imaging system 100 can have a handheld imaging probe 140. The handheld imaging probe 140 can have a probe housing 141 with a distal end. The terms “distal” and “front” are used interchangeably in this disclosure. Both “distal end” and “front end” means the end closest to the cornea 203 of the eye. The handheld imaging probe 140 can comprise a contact optical window 202 disposed at the distal end of the probe housing 141. The optical window 202 is configured to be in contact with a cornea 203 of the eye, as disclosed in U.S. Pat. No. 9,155,466. The optical window 202 is configured to be in contact with the cornea 203 directly or through a disposable and optically clear cap. An index matching gel can be applied to the cornea 203 and/or the optical window 202 and/or the disposable cap. In order for the first imaging module 120 to have a wide field of view, the use of the optical index matching gel between the optical window 202 and the cornea 203 helps to eliminate significant amount of optical aberrations originated from the cornea 203 of the eye. The detail of the disposable cap may be as disclosed in U.S. patent application Ser. No. 15/007,101, titled: “A DISPOSABLE CAP FOR AN EYE IMAGING APPARATUS AND RELATED METHODS” and filed on Jan. 26, 2016, which is incorporated by reference in its entirety. The term “in contact” is defined herein to include in contact directly, in contact through an index matching gel, and in contact through a disposable cap.

In use, the optical window 202 may be placed in contact with the cornea 203 with slight pressure to obtain a wide field of the view of the retina 201 through the pupil. Accordingly, the optical window 202 may have a front concave surface 202a with a radius of curvature closely matching a curvature of the cornea 203 of the eye. In some embodiments, the front concave surface 202a of the optical window 202 has a radius of curvature of between 3 mm and 18 mm. For example, the front concave surface 202a of the optical window 202 can have a radius of curvature of between 5 mm and 15 mm. Values outside the above range are also possible. The radius of curvature of the concave front surface of the optical window 202 can be changed depends on the need of the application.

As shown in FIG. 2B, the distal surface of the optical window 102 can comprise the front concave surface 202a, and a small flat, distally facing ring surface 202b extending radially outward from the concave front surface 202a. All of the surfaces can be optically polished. The optical window 202 is optically transparent and can comprise materials such as optical glass. The distal end of the probe housing 141 extends around the edge of the optical window 202. The distal end of the probe housing 106 has a smooth ridge to prevent injury to the patients during the operation and to protect the optical window 202 from scratching by hard foreign objects. The small flat surface 202b, in the form of a circular ring, may be disposed on the front peripheral area of the optical window 202. This small flat ring 202b may be near and/or extend from the side of the optical window 202 to or near to the edge of the front concave surface 202a of the optical window 202. A small alignment ring or edge can be disposed into the probe housing 141. In various embodiments, the dimensions of the alignment ring can be configured to match the distally facing flat surface 202b of the optical window. The distally facing flat surface 202b of the optical window 202 can assist in the alignment of the optical window 202 and the probe housing 141, thus reducing aberration from misalignment. The distally facing flat surface 202b can further provide a tight fit between the optical window 202 and the probe housing 141.

When the imaging probe 140 comes to in contact with the cornea 203 of the eye directly or through a disposable cap, the imaging apparatus 100 can be used to image the posterior segment 201, or the retina, of the eye, in the form of a contact microscope. The contact OCT imaging system 100 is advantageous for pediatric application since the babies are often not following the instruction of the operators. For pediatric application, the non-contact type OCT imaging system does not work well at babies because the non-contact OCT imaging system need a cooperative object who can follow the instructions of the operator. The contact type OCT imaging system can overcome the problems of the non-contact system and work well with babies.

Moreover, the contact type OCT imaging system 100 can further result in a super wide field of view. For example, the contact OCT imaging system 100 can have a field of view that is 3 to 5 times of a conventional non-contact OCT imaging system. In some embodiments, the contact OCT imaging system 100 can have a field of view of between 60 degrees to 120 degrees in a single volume acquisition. In some embodiments, the contact OCT imaging system 100 can have a field of view of 130 degrees in a single volume acquisition. The microscope type optical design of the contact OCT imaging system 100 can result in a wider field of view than the telescope style optical design of the conventional non-contact OCT imaging systems for imaging the posterior segment of the eye.

One of the problems of non-contact OCT imaging systems is the need for sophisticated automatic (or smart) adjustment for a reference arm. In general, an OCT imaging system has an axial resolution around 6 μm. The change in axial distance between the OCT imaging system and the eye of the patient causes the vertical motion in OCT images. The distance between the OCT imaging system and the eye of the patient has to be kept constant to the range of tens of microns in order to see the live OCT images stabilized to an acceptable level. To keep the OCT images even in the picture frame, which is related to the working range of the axial distance, the distance has to be maintained to be within less than 1-2 mm. For a non-contact OCT imaging probe, which may be about 3 lbs. to 6 lbs. and away from the eye, it is very difficult to align the non-contact OCT imaging probe precisely with an eye of an infant or a small child. Conventionally, it typically takes about 20 minutes or more for doctors/operators to get alignment right and be able to take an OCT image since often the OCT images may only show up for a couple of seconds and move out. The eye of a child may move, and the hand of the doctor/operator may move as well. It is difficult to automatically compensate and stabilize such motions in the OCT images by the reference arm adjustment.

The contact OCT imaging system 100 can better stabilize the OCT images during the operation and increase the signal-to-noise ratio of the OCT images than the conventional non-contact OCT imaging systems. The contact OCT imaging system 100 is placed in contact with the cornea of the eye; such arrangement can minimize the possible change of distance between the eye (more precisely the retina for the posterior segment imaging) and the OCT imaging system 100 to a fraction of 1 mm, thus reducing the vertical motion in OCT images. Therefore, during an OCT imaging procedure, the OCT image is easily kept within the picture frame. As soon as the OCT imaging system 100 is in contact with the cornea, the doctors/operators may immediately see the live OCT image without the need to struggle for alignment. The live OCT image may still move in the picture frame constantly because the distance control is still not to the level of tens of microns. However, combining with high speed imaging technique, the quality of the OCT image can be significantly improved, and the time to acquire the OCT image can be significantly reduced.

In addition, the OCT imaging system 100 can minimize the lateral alignment requirement between the patient's eye and the optical system of the OCT imaging system 100. Because the shape of the contact optical window 202 is designed to fit closely with the shape of the cornea, it is easier to align the contact optical window 202 with the cornea when they are in contact. The contact optical window 202 can also reduce the free motion of the eye ball too, and helps to stabilize images in its lateral motion. The OCT imaging system 100 can further minimize the optical aberration caused by either the misalignment of the eye with the OCT imaging system 100, or the aberration from the eye, by using the index matching gel in the space between the eye and the contact optical window 202. Such reduction helps to increase the signal to noise ratio of the OCT images because more light could be couple to the optical fiber from the eye when aberration is reduced. The contact optical window 202 can widen the field of the view of the OCT imaging system 100 and enable the imaging of peripheral area of the posterior segment 201, the retina, where the conventional non-contact type OCT imaging systems cannot reach.

Referring to FIG. 2A, the handheld imaging probe 140 of the OCT imaging system 100 can comprise a first imaging module 120 and an OCT imaging module 110. For example, the first imaging module 120 can be a color imaging module, or a full field imaging module. In general, the first imaging module 120 can be configured to capture a color image of an eye, or a photograph of the eye. The first imaging module 120 can have a first illumination path and a first imaging path, where the first imaging path is separated from the first illumination path. The first imaging module 120 can comprise a first light source 221 disposed inside the imaging probe 140 to illuminate the eye through the independent illumination optical path. The first imaging module 120 can comprise imaging and focusing optics and an image sensor 210. The image sensor 210 is configured to receive reflected light from the eye through the separated imaging path in the first imaging module 120. The first illumination path is outside the first imaging path, and the first imaging path does not overlap with the first illumination path. The separation of the illumination path and the imaging path can reduce the scattered light from the cornea and widen the field of view of the OCT imaging system 100 when imaging the posterior segment of the eye, the retina. Otherwise, the scattering light from the cornea is much stronger than the reflected light from the retina, and the achievable field of view of the imaging system may be limited.

The first imaging module 120 of the OCT imaging system 100 can comprise an imaging lens 204. The imaging lens 204 can be positioned behind the optical window 202 and optically aligned with the optical window 202. The first imaging module 120 can further comprise a light conditioning element 218. The light conditioning element 218 can comprise a multi-segment (e.g., reflective and/or refractive) surface configured to receive a first light beam from the first light source 221 and directional control the first light beam to illuminate the eye in a desired way to result in a wide field of view of the first imaging module 120.

The imaging lens 204, which may include one or multiple lens elements, is positioned behind the optical window 202, on the opposite side of the optical window 202 as the eye, and optically aligned with the optical window 202. The optical axis of the optical window 202 and imaging lens 204 may, for example, be substantially aligned with the optical axis of the eye in some cases but not all. For example, the practitioner may examine the eye in a manner that the optical axis of the first imaging system 120 is substantially aligned with the optical axis of the eye; however, in some cases, the practitioner may tilt the handheld imaging probe 140 such that these axes are not aligned. Although the radius of the curvature for the frontal surface of the optical window 202 is chosen to closely match that of the cornea, the back surface of the optical window 202 may be flattened out slightly depending on the design of the first illumination path. The optical window 202 may be made from the same or different optical materials as the imaging lens 204. The curvature of the frontal surface of the imaging lens 204 may be the same as that of the back surface of the optical window 202, or different. The back surface of the imaging lens 204 may be either spherical or non-spherical to obtain desired result for the images. In some embodiments, a small gap of air or other material is placed between the optical window 202 and the imaging lens 204, although the two optical components may be in contact in certain areas or even bonded or affixed together with adhesive.

In some embodiments, the optical imaging system 120 may further include a first set of relay lenses 205 configured to form a secondary image 208 of the eye near a back focal plane of the first set of relay lenses, a second set of relay lenses 209 configured to project the secondary image 208 to infinity with a front focal plane positioned near the back focal plane of the first set of relay lenses. In various embodiments, a first focusing lens 211 is positioned near the back focal plane of the second set of relay lenses and configured to deliver light from the eye to the image sensor 210. For example, the first focusing lens can be a set of miniature focusing lenses. In some embodiments, the image sensor 210 and the first focusing lens 211 can be disposed in a miniature camera module. The miniature camera module comprising the first focusing lens 211 and the image sensor 210 has a format no more than 1/2.2 inches or 1/3.2 inches with a focal length of about 4 mm or less, for example between about 4 mm and 2 mm or 4 mm and 3 mm, etc. The view angle for the miniature lens or lenses may be 75° or less with a sensor appropriately sized based, for example, on the focal length of the miniature lens. The camera module, which includes the sensor chip 210 and the first focusing lens or lenses 211, is about 8.5×8.5 mm, or between 10 mm×10 mm and 5 mm×5 mm or smaller, for example. In some embodiment, for example, the first focusing lens or lenses 211 have aperture sizes between about 0.8 mm and 1.5 mm while the first and second relay lenses 205, 209 have aperture sizes of about 20 mm, for example between about 30 mm and 10 mm or 25 mm and 15 mm in some embodiments. The first imaging system 120 may gather light reflected from the posterior segment or more specifically the retina of the eye 201. The light passes through the center of an iris opening and the crystalline lens 207 of the eye, and forms a real image (of the posterior segment or retina) at the secondary image plane 208. As discussed above, the imaging lens 204 may include single or multiple lenses, with spherical or non-spherical surfaces. In some embodiments, the secondary image plane 208 is located near the back focal plane of lens 205. In some embodiments, a relay lens 209 may be used to project the image from the secondary image plane 208 to infinity when the front focal plane of the lens 209 is also placed near the secondary image plane 208. The image sensor 210, either in form of CCD, CMOS or other types, with its own miniature focusing lenses 211, may be positioned near the back focal plane of the lens 209 along the optical axis of the optical imaging system 120. The miniature lenses 211 may include multiple optical lenses. In some embodiments, the image sensor 210 has an active area that is about 6.2 mm×4.6 mm or, for example, between about 8 mm and 4 mm×6 mm and 3 mm or between about 7 mm and 5 mm×5 mm and 4 mm. Accordingly, in various embodiments the active areas of the sensor 210 are about ¼ of the aperture size of the relay lenses 205, 208 or for example between about 0.4 and 0.2 or 0.5 and 0.1 the size thereof. In various embodiments, the first focusing lens or lenses 211 are built with a circular optical aperture (iris) 212, which may be located between the first focusing lens or lenses 211 or formed by an aperture plate in front of the first focusing lens or lenses 211. In certain embodiments such location of the optical aperture 212 reduces optical aberration. The first focusing lens or lenses 211 may not only relay the image of the retina 201 to the image sensor 210, but also form an entrance pupil 243 for the first imaging system 120 near the anterior surface of crystalline lens 207 when the aperture 212 becomes the aperture of the entire optical imaging system 120. This special arrangement helps to eliminate significant amount of scattering light from the anterior chamber of the eye and the optical elements in the first imaging module 120.

In some embodiments, the optical lenses in the first imaging path of the first imaging module 120 can be achromatized for the light beams within the wavelength range of the first light source 221. The optical lenses forming the first imaging module 120, from the distal end of the first imaging module, which includes the contact optical window 202, to the front of the imaging sensor 210, which includes the focusing lens 211, can be achromatized for the light beams of multiple wavelengths within the wavelength range of the first light source 221 in the first imaging module 120. Additional considerations may be needed in order to compensate the residual chromatic aberrations from the eye itself. Here the achromatization means the design to minimize the optical aberrations for multiple light wavelengths within the working wavelength range, not just one wavelength, as well as for the full field of view of the first imaging module 120. For example, the optical lenses forming the first imaging module 120 can be configured to minimize the optical aberrations at wavelengths of 470 nm, 550 nm and 650 nm for the working light wavelength range of 450 nm to 700 nm, and minimize the aberrations for the field of view from 0 degree (on optical axis) to 130 degree as well.

The first imaging module 120 can comprise a first illumination sub-module, which comprises the first light source 221, optical fiber bundles 220, the light conditioning element 218, and a periphery portion of the optical window 201. The first illumination sub-module forms the first illumination path, which is separated in space from the first imaging path. The first light beam may be emitted from the light source 221 and injected into the optical light conditioning element 218 positioned behind the peripheral portion of the optical window 202 though optical fiber bundles 220. The first illumination path is extending longitudinally forwardly and radially inwardly from the first light source 221, to optical fiber bundles 220, to the light conditioning element 218, to the periphery portion of the optical window 201, and to the posterior segment 201, the retina, of the eye for posterior imaging. The first imaging module 120 can comprise a first imaging sub-module, which comprises a central portion of the optical window 202, the imaging lens 204, the first relay lens 205, the second relay lens 209, the first focusing lens 211 and the image sensor 210. The first imaging sub-module forms the first imaging path for the reflected light from the eye. The first imaging path is extending longitudinally backwardly from the retina 207 to the central portion of the optical window 202, to the imaging lens 204, to the first relay lens 205, to the second relay lens 209, to the first focusing lens 211, and to the image sensor 210.

To obtain high quality images, proper illumination is provided through the proper portion of the natural opening of the eye while avoiding the first imaging path. In particular, illumination is provided through the peripheral regions of the eye pupil while the light scattered back from the posterior segment 201, the retina, of the eye will pass through the central portion of the eye pupil which eventually forms the first image by the image sensor 210. This approach reduces backscatter from the central portions of the pupil, which would degrade the image of the retina obtained by light reflected from the retina also passing through the pupil. Since the eye is a complicated biological organ with its own special optical systems, the scattering and reflection from the eye in combination with its small aperture cause significant difficulties in obtaining a high quality image. In particular, the reflection and scattering from the eye cause glare and haze, which obscures the images acquired by an eye imaging apparatus. Thus the images from the posterior segment of the eye with a wide field of view often exhibit a layer of strong haze or glare. This problem is especially acute for the patients with dark pigmentation in the eyes. Providing illumination through certain regions of the eye as described herein, however, can reduce this backscatter and reflection and the resultant haze and glare. Therefore, the first imaging module 120 can further achieve a wide field of view.

FIG. 2C is a perspective view of the light conditioning element 218 in the first imaging module of the OCT imaging system 100. The detailed description of the light conditioning element 218 is disclosed in U.S. Pat. No. 9,155,466, which is herein incorporated by reference in its entirety. As shown in FIG. 2B, the light conditioning element 218 has a hollow ring shaped body that is configured to be disposed about or around the imaging lens 204. The body of light conditioning element 218 may comprise a hollow truncated cone-shaped solid structure comprising of solid optically transmissive or transparent material. However, in certain embodiments the light conditioning element comprises opaque material. Accordingly, the light conditioning element may comprise glass, plastic, ceramic, metal or combinations thereof. Other materials may also be employed. This shape may be characterized as a hollow and ring-shaped and frusto-conical. The front surface and the back surface as well as cross sections orthogonal to the length are in the shape of a circular ring. The back surface has a larger lateral extent, e.g., inner and outer radii than the front surface. At least one of the surfaces of the light conditioning element 218 comprises a multi-segment surface having multiple reflective and/or refractive segments. The different segments in the multi-segment surface may have different orientations, different shapes, different coatings, or any other different configurations. The light conditioning element 218 may distribute light received from the light source into different portions as a result of the different segments in the multi-segment reflective and refractive surface. In some embodiments, light from the light source that is reflected from the multi-segment light conditioning element is distributed the light into different portions by total internal reflection and possibly refraction of the multi-segment surface. In some embodiments, the light conditioning element distributes the light from the light source into different portions for example by total internal reflection and refraction of the multi-segment surface.

The light conditioning element 218 may provide a light channel for propagation of light. In some embodiment the light conditioning element 218 is configured to direct a first portion of light from the inner edge of the light channel to a first area of a retina of the eye including an optical axis of the optical imaging system. In various embodiments, the first area comprises one-third of the field of view of the optical imaging system. When the optical axis of the optical imaging system is aligned with the optical axis of the eye, the first area is the central area of the retina of the eye. In various embodiments, the light conditioning element is configured to direct more than 50%, 60%, 70% and 80% of the light exiting from the inner edge of the light channel to the first area of the retina. The light conditioning element is also configured to direct a second portion of light from the outer edge of the light channel to a second area of a retina of the eye away from the optical axis and on an opposite side of the optical axis from the outer edge of the light channel from which the light is ejected. The second area is farther from the optical axis than two-third of the field of view of the imaging system. When the optical axis of the optical imaging system is aligned with the optical axis of the eye, the second area is the peripheral area of the retina. In various embodiments, the light conditioning element is configured to direct more than 50%, 60% and 70% of 1 the light exiting from the outer edge of the light channel to the second area of the retina.

The first light source 221 can be a visible light source, for example, with a wavelength from 450 nm to 700 nm in some embodiments. The first imaging module 120 can be used to obtain a color full field image of the eye. The color full field image is preferred by doctors since it provides more information than a black and white image. However, in order to obtain a color image by using a visible light source, the dilation of the eye is required. In some other embodiments, the first light source 221 can be a near infrared (NIR) light with a wavelength range from 700 nm to 840 nm. When the first light source 221 is a NIR light source, the eye does not need to be dilated, which may make a child more comfortable during the OCT imaging session. The NIR light beam of the NIR light source has less scattering than the visible light beam because the longer wavelength light cause less scattering. Therefore, the OCT imaging system may get better and clearer OCT images by using the NIR light source than using the visible light source.

Referring back to FIG. 1 and FIG. 2A, the OCT imaging system 100 can comprise an OCT imaging module 110 (an integrated OCT Imaging Module, or a removable OCT Imaging Module), which can provide OCT imaging in concurrent with the color imaging at same time. In some embodiments, the OCT imaging module 110 may is permanently integrated with the first imaging module 120. A second light beam from a broadband source 250, which is in the OCT engine 131 disposed in a console 130, can be carried to the OCT imaging module 110, through an optical fiber 103 to form a sample arm for the OCT interferometer (not shown). The optical fiber 103 can be a single mode fiber, either in the form of regular fiber or polarization maintaining fiber. The polarization maintaining fiber can reduce the effect of the shift of polarization state for the emitting light when the fiber 103 is being bended or twisted during the operation. The details of the polarization maintaining fiber will be discussed below. An optical coupling lens 104 mounted in an either mechanical or electrical focus adjustment mechanism can be used to form a collimated light beam.

A beam splitting dichroic mirror 102, which can be configured to reflect light in the wavelength longer than 700 nm, can be inserted into the first imaging path of the first imaging module 120. The first imaging module 120 provides color imaging capability in the visible light spectrum, from 450 nm to 700 nm. The first light beam of the first imaging module 120 can pass through the dichroic mirror 102 while the second light beam from a second light source for the OCT imaging module can be reflected almost entirely. The second light source is an OCT light source. For example, the second light source can be a broadband light source with a wavelength range from 800 nm to 1200 nm that is disposed in the console 130. The second light source can be a swept source laser that is disposed in the console 130 in some embodiments.

The OCT imaging module 110 can comprise a second illumination path and a second imaging path. The OCT imaging module 110 can use some of the same optical components used in the first imaging module 120 to perform its illumination and imaging functions. The OCT imaging module 110 can be configured to construct a cross-sectional view of the eye. The OCT imaging module 110 can comprise a beam splitting dichroic mirror 102 which can be configured to split the light beam in the imaging path for first imaging module 120 and OCT imaging module 110, and a scanning mirror, for example, a scanning MEMS mirror 112. The mirror driver or drivers can be connected to a scanning MEMS mirror controller 134 disposed in the console 130 by a data link 136. The OCT imaging module 110 and first imaging module 120 share same optics from the beam splitting dichroic mirror 102 to the distal of the optics toward the patient's eye. For example, the OCT imaging module 110 and first imaging module 120 share the beam splitting dichroic mirror 102, the first relay lens 205, the imaging lens 204, and the optical window 202 as shown in FIG. 2A. The beam splitting dichroic mirror 102 can be disposed in the first imaging path and configured to transmit the first light beam of the first light source 221 and reflect the first portion of a second light beam from the second light source (not shown). In some embodiments, the scanning MEMS mirror 112 can be configured to scan a first portion of the second light beam from the second light source, where the scanning MEMS mirror disposed outside the first illumination path and the first imaging path. The optical first imaging module 120 can be configured to provide registry of an imaging location of the OCT imaging module 110.

The OCT imaging module can comprise a second focusing lens or lenses 114 configured to perform focus adjustment for the OCT imaging module. The OCT imaging module can further comprise one or mirrors to manage the optical path of the second beam as shown in FIG. 2A. The second light beam can be focused by the second focusing lens or lenses 114, through the beam slitting dichroic mirror 102, on to an area near the secondary imaging plane 208 of the first imaging module 120. From there, the second light beam can be directed by the imaging optics (for example, the contact optical window 202, the imaging lens 204 and the first relay lens 205) in the first imaging module 120 to the targeted portion of the eye, for example, the posterior segment 201. The imaging optics along this shared optical path can be designed and achromatized for working in both the wavelengths for visible first imaging module 120 and OCT imaging module 110. Therefore, the first imaging 120 and OCT imaging module 110 can share the same or approximately same secondary image plane 208 as shown in FIG. 2A. The optics for the OCT imaging path can be also designed such that the scanning mirror 112 can be located in a plane as an optical conjugate for the entrance pupil 243 of the first imaging module 120. The entrance pupil 243 can be located near the iris plane of the eye, which is separated from the illumination light path of the first imaging module and located near the central portion of the eye iris. In some embodiments, a real image of an aperture of the OCT imaging module is positioned near an anterior surface of the crystalline lens of the eye when the optical window is in contact of the cornea. When the scanning mirror 112 is rotated, the mirror 112 can direct the focused light beam to different locations in the posterior segment 201 of the eye. With different type of scanning patterns, the light beam can illuminate different parts of posterior segment 201 of eye in a timely and consecutive fashion.

Because the first imaging module 120 has its own focusing mechanism which can activated when the posterior segment or retina 201 is out of focus, both the first image module 120 and the OCT image imaging module 110 can be configured to be focused at same time. To do that, part of the focusing lens group 114 can be configured to be movable, which helps to re-focus the OCT light beam on to the target if necessary. The movable focusing lens group 114 can be driven electrically with the driver electronics built on the board of driver electronics for scanning mirror 112 or manually. When the focusing lens group 114 is driven electrically, the motion of the focusing lens group 114 can be configured to synchronize with the motion of the focusing lens 211 of the first imaging module 120 such that the focusing action for both imaging modalities could be performed at same time with just one action. The focus of the OCT light beam can also be achieved by moving the couple lens 104 with its driving mechanism electrically with the driver electronics built on the board of driver electronics for scanning mirror 112, or manually.

In some embodiments, the OCT imaging system 100 can further comprise a third light source 253, which is an aiming light. The third light source is disposed in the console 130. A beam combiner 252 is also disposed in the console 130, and the beam combiner 253 is configured to couple both the second light source 250 and the third light source 253 to the handheld imaging probe 140 through the optical fiber 103. The light from the optical fiber 103 not only includes the second light beam from the broadband light source 250 for OCT imaging, but also a third light beam as the aiming light with narrowband wavelength range. In some embodiments, the wavelength of the third light source 253 can be between the second light source 250 and the first light source 221 for color imaging. For example, the aiming light source can have a wavelength of 680 nm, 700 nm or 740 nm. The third light beam can have a third illumination path and a third imaging path, wherein the third illumination path is the same as the second illumination path of the OCT imaging module and the third imaging path is the same as the first imaging path of the first imaging module. The beam splitting dichroic mirror 102 can be configured to partially reflect and partially transmit the third light beam. The aiming light beam is relayed to the posterior segment 201 of the eye with the same optics of the OCT imaging module 110 and focused as at exactly the same spot, while the reflected aiming light beam is partially transmitted through the beam splitter 102 and then eventually projected to the image sensor 210 by the same optics as in the first imaging path.

The track of the aiming light beam is then shown on the first image, which not only provides visualization of the OCT scanning pattern in real time, but also a focusing status of the OCT scanning beam on the posterior segment 201. During the process of adjusting the first focus of the first imaging module 120 to give a clear color image, the linewidth of the aiming light beam seen on the image is also changed. The linewidth of the aiming light beam then can be measured in real time from the color image and provides feedback for the adjustment of the second focus for OCT imaging module. The feedback of the linewidth of the aiming light beam can be used to control the second focusing lens, for example, and the movable lens or lenses 114, or the coupling lens 104. As the result, the focus for both the first imaging module 120 and the OCT imaging module 110 can be synchronized in real time. Thus, the first adjustment of the first focus lens 211 and a second adjustment of the second focus lens are synchronized through the feedback of the linewidth of the aiming light beam.

As discussed above, the optical lenses in the first imaging path of the first imaging module 120 can be achromatized for the light beams within the wavelength range of the first light source 221. Because all of optical materials have their unique properties of color dispersion, if the first imaging module 120 is not achromatized, then the images formed by different color of light beams will not be superimposed into one image on the imaging sensor 210, causing reduction in contrast of the image significantly. Conventionally, it is not realized that the OCT imaging system has even more severe problem of color dispersion than the first imaging module. In general, an OCT imaging system comes with a broadband light source which can have an optical wavelength range spanning up to 100 nm. If the OCT imaging system is not achromatized, the light beams from different wavelengths, even if they traveled through the same optical pathway, will exhibit different total optical paths in the sample arm and result in different interference patterns when interfered with the reference arm in the fiber interferometer. Most of the conventional OCT imaging systems are trying to solve the problem by using compensation module in the reference arm. For example, the compensation module can contain the same materials of the same thickness in the sample arm. However, for the OCT imaging systems, the light beams at different field of view (view angles) can also have different total optical paths even though the light beams go through the same optical materials. In general, the lenses have different thickness from their centers to the edges. When the light beams pass through different portions of the lenses, the total optical paths have different percentage of space (air) and glass mixtures for different field of view in the full imaging system. Because the dispersion compensation module is a fixed one, which can be designed to match the glasses type and thickness for the light beam along the optical axis in the imaging path, but the compensation module may not be correct for the light beam traveling through different part of the lenses, such as off-axis light beams. Moreover, the eye has optical dispersion which is not corrected. The lens in the imaging path have to be achromatized such that not only the aberrations caused by the lens materials are compensated, but also the optical dispersion from the eye is compensated.

In some embodiments, the optical lenses in the second imaging path of the OCT imaging module 110 can be achromatized for the light beams within the wavelength range of the OCT light source 250 for optical dispersion, from the eye all the way to the front end of the fiber 103. The optical lenses forming the OCT imaging module 110 from the distal end of the second imaging path, which includes the optical window 202, to the front end of the optical fiber 103, which is the optical coupling lens 104, can be achromatized for the light with wavelengths used in the OCT imaging module. The optical lenses in the second imaging path are further configured with additional consideration for the need to compensate the residual chromatic aberrations from the eye itself. Here the achromatization means the design to minimize the optical aberrations for multiple light wavelengths within the working wavelength range, not just one wavelength, as well as for the full field of view of the OCT imaging module 110. For example, the optical lenses in the second imaging path are configured to minimize the optical aberrations at wavelengths of 1000 nm and 1080 nm for the working light wavelength range of 980 nm to 1100 nm, as well as for the field of view from 0 degree (on optical axis) to 130 degree.

In some other embodiments, the achromatization can be performed to minimize the axial color aberration for the selected light wavelengths for the full field of view of the OCT imaging module 110. The optical path difference for the light beams transmitting within the same portion of the optical lenses but with different optical wavelengths can be minimized to an allowable amount, and the same allowable amount of optical path difference can be applied to light beams transmitting through different portions of the optical lenses. For example, the light path difference for the light beams with optical wavelengths of 1000 nm and 1090 nm can be minimized to 2 nm for light beams transmitting along the optical axis and minimized to around 2 nm for light beam with the field of view of 130 degrees.

In some embodiments, the optical lenses in the first imaging path of the first imaging module 120 can be achromatized for optical dispersion for the wavelength range of the first light source 221, from the eye all the way to the imaging sensor 210. Therefore, the optical lenses in the shared portion of the first imaging path and the second imaging path, from the distal end of the imaging probe 140 to the dichroic beam splitting mirror 102, can be achromatized for optical dispersion of the working optical wavelengths of both the first imaging module 120 and the OCT imaging module 110. The optical lenses shared by both the first imaging module 120 and the OCT imaging module 110, for example, the optical contact window 202, the imaging lens 204 and relay lens 205, can be achromatized for both optical wavelength bands in the first imaging module 120 and the OCT imaging module 110.

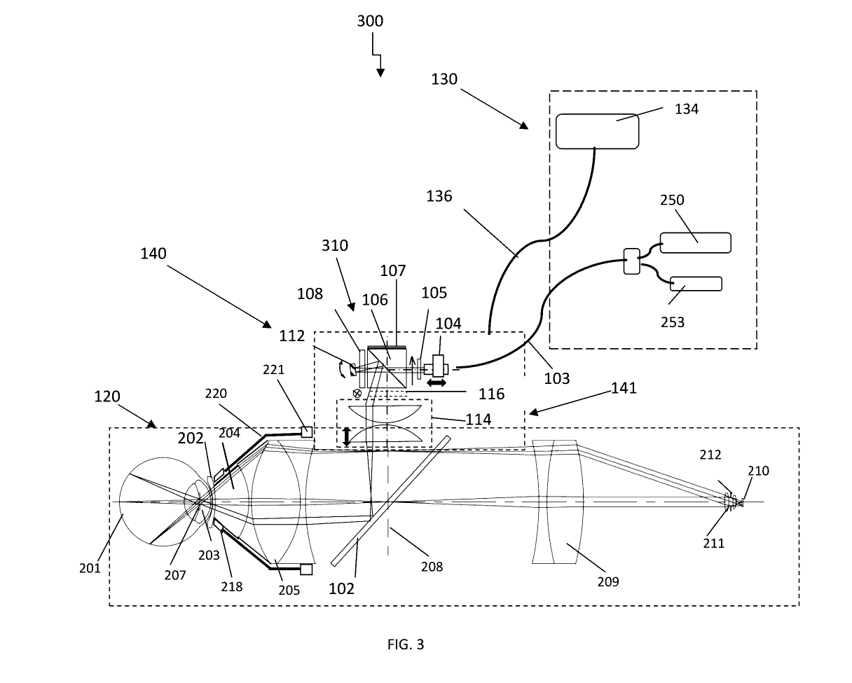

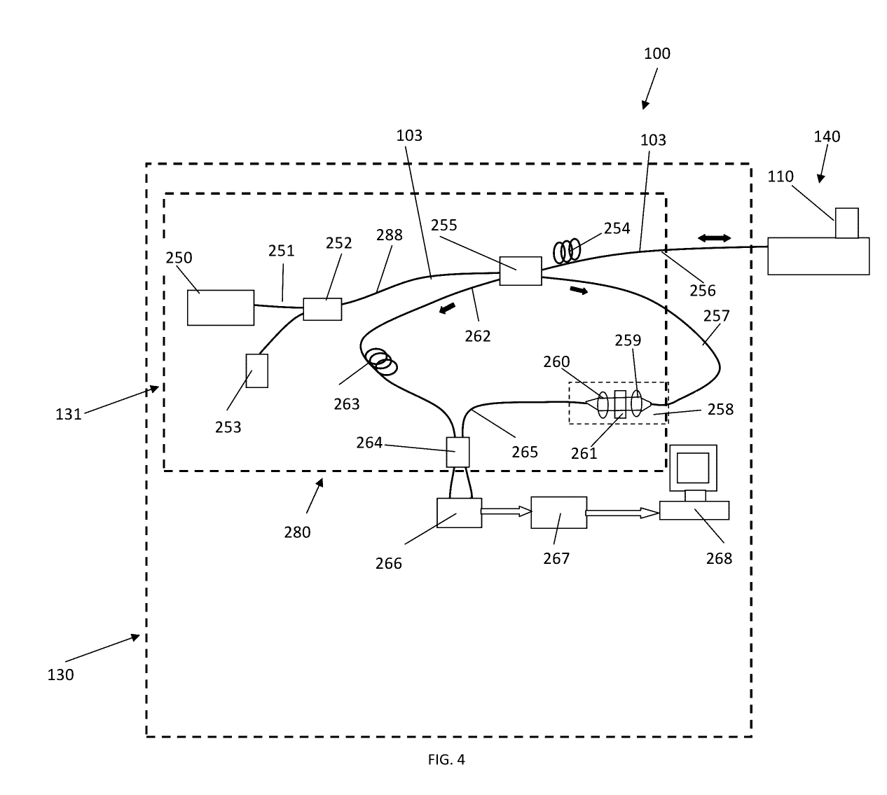

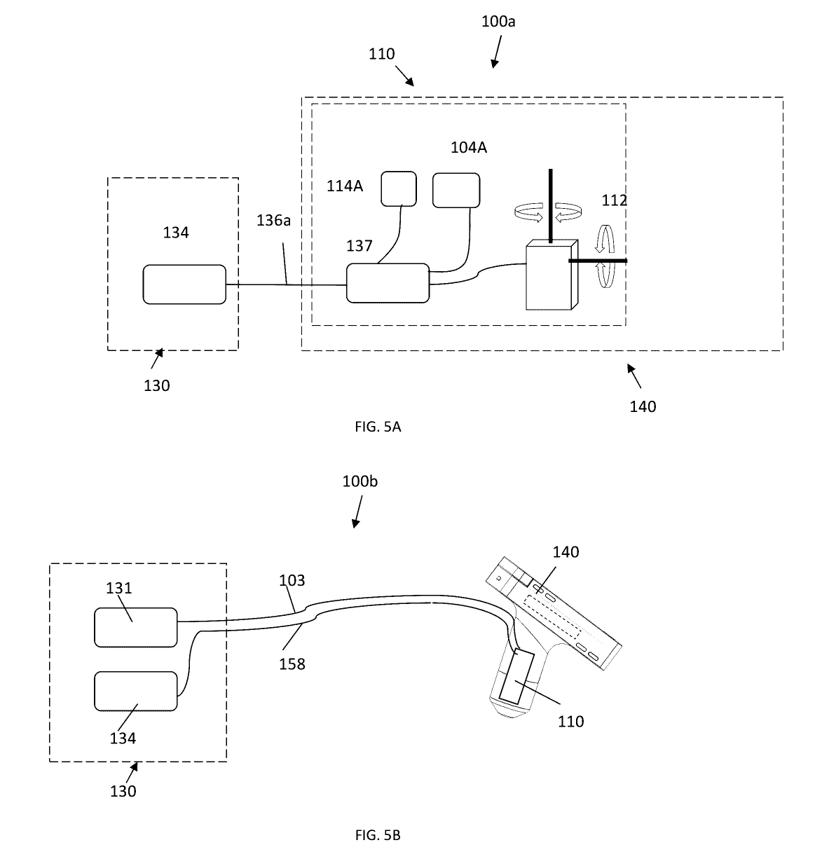

FIG. 3 is a schematic of another example optical design of an OCT imaging system 300 with a wide field of view where a posterior segment 201 of an eye is imaged in another embodiment. A second light beam from a broadband source 250 disposed in a console 130 is carried to the OCT imaging module 310, through an optical fiber 103 to form a sample arm for the OCT interferometer (not shown). The optical fiber 103 can be a single mode fiber, either in the form of regular fiber or polarization maintaining fiber. An optical coupling lens 104 mounted in an either mechanical or electrical focus adjustment mechanism can be used to form a collimated light beam.

A broadband linear polarizer 105 can be used to clean up the undesired light in other polarization state to form a p polarization of light beam. The second light beam can be then injected into a polarization beam splitter (PBS) 106 which allows the transmission of the p polarized beam and reflects s polarized beam. A broadband quarter-wave plate 108, which can be located next to the PBS 106, coverts the p polarized beam into a circularly polarized light beam, for example left-hand or right hand circular polarization. The scanning mirror 112 can be coated with highly reflective coating, either metallic or dichroic. In some embodiments, the scanning mirror 112 can be a MEMS mirror driven in two orthogonal directions by a mirror driver, or two MEMS mirrors with their rotation axes arranged in two orthogonal directions and by two mirror drivers. The mirror driver or drivers can be connected to a scanning MEMS mirror controller 134 disposed in the console 130 by a data link 136. After the circularly polarized light beam is reflected from the scanning mirror 112, the direction of rotation is changed, for example, from left-hand polarization to right-hand polarization or from right hand polarization to left hand polarization, before entering the 108 second time in opposite direction. The quarter-wave plate 108 then converts the circularly polarized light beam into linearly polarized light beam, this time in the form of s polarization. The s polarized light beam can be reflected by the PBS 106 in orthogonal direction and projected into the focusing lens group 114. In another embodiment, a second broadband quarter-wave plate 116 can be used between the PBS 106 and focusing lens group 114 to convert the linearly polarized beam into a circularly polarized beam. The light beam often can be in the collimated state.

The second light beam can be focused by a second focusing lens or lenses 114 on to an area near the secondary imaging plane 208 of the first imaging module 120. From there, the second light beam can be directed by the imaging optics in the first imaging module 120 to the targeted portion of the eye, for example, the posterior segment 201. The imaging optics along this shared optical path can be designed and optimized for working in both the wavelengths for visible first imaging module 120 and OCT imaging module 310. Therefore, the first imaging 120 and OCT imaging module 310 can share the same or approximately same secondary image plane 208 as shown in FIG. 3. The optics for the OCT imaging path can be also designed such that the scanning mirror 112 can be located in a plane as an optical conjugate for the entrance pupil 243 of the first imaging module 120. The entrance pupil 243 can be located near the iris plane of the eye, which is separated from the illumination light path of the first imaging module and located near the central portion of the eye iris. In some embodiments, a real image of an aperture of the OCT imaging module 310 is positioned near an anterior surface of the crystalline lens of the eye when the optical window 202 is in contact of the cornea 203. When the scanning mirror 112 is rotated, the mirror 112 can direct the focused light beam to different locations in the posterior segment 201 of the eye.

The light scattered by the tissue in the eye at the spot of illumination at any given time, for example, the posterior segment 201 of the eye, can be collected by the same optics, including the imaging optics (for example, the contact optical window 202, the imaging lens 204 and the first relay lens 205), beam slitting dichroic mirror 102, focusing lens group 114, PBS 106 and quarter-wave plate 108, and returns to the scanning mirror 112 in the form of circularly, linear, partially, or randomly polarized light beam instantaneously. The scanning mirror 112 can then reflect the light beam back through quarter-wave plate 108, in the form of linearly p polarized, partially or randomly polarized beam, going through the PBS 106 again. With the help of the fiber coupling lens 104 and the broadband linear polarizer 105, the light can be injected into the optical fiber 103 and transmitted back to the OCT engine 131, in the same polarization state, especially in the case of polarization maintaining fiber. Some of the unwanted straight light, both in the projection light path and returning light path in the OCT imaging module 310, can be absorbed by the absorption layer 107 coated on one of surface of the PBS 106, resulting in better signal to noise performance.

Both the first image module 120 and the OCT image imaging module 310 can be configured to be focused at same time. The focusing lens 114, or part of the focusing lens group 114 can be configured to be movable, which helps to re-focus the OCT light beam on to the target if necessary. The movable focusing lens group 114 can be driven electrically with the driver electronics built on the board of driver electronics for scanning mirror 112 or manually. When the focusing lens group 114 is driven electrically, the motion of the focusing lens group 114 can be configured to synchronize with the motion of the focusing lens 211 of the first imaging module 120 such that the focusing action for both imaging modalities could be performed at same time with just one action.

In some embodiments, the OCT imaging system 300 can further comprise a third light source 253. The light from the optical fiber 103 not only includes the second light beam from the broadband light source 250 for OCT imaging, but also a third light beam as the aiming light with narrowband wavelength range. In some embodiments, the wavelength of the third light source 253 can be between the second light source 250 and the first light source 221 for color imaging. For example, the aiming light source can have a wavelength of 680 nm, 700 nm or 740 nm. The third light beam can have a third illumination path and a third imaging path, wherein the third illumination path is the same as the second illumination path of the OCT imaging module and the third imaging path is the same as the first imaging path of the first imaging module. The beam splitting dichroic mirror 102 can be configured to partially reflect and partially transmit the third light beam. The aiming light beam is relayed to the posterior segment 201 of the eye with the same optics of the OCT imaging module 310 and focused as at the exactly same spot, while the reflected aiming light beam is partially transmitted through the beam splitter 102 and then eventually projected to the image sensor 210 by the same optics as in the first imaging path. The track of the aiming light beam is then shown on the first image, which not only provides visualization of the OCT scanning pattern in real time, but also a focusing status of the OCT scanning beam on the posterior segment 201. The linewidth of the aiming light beam then can be measured in real time from the color image and provides feedback for the adjustment of the second focus for OCT imaging module. The first adjustment of the first focus lens 211 and a second adjustment of the second focus lens are synchronized through the feedback of the linewidth of the aiming light beam.

In some embodiments, the optical lenses in the second imaging path of the OCT imaging module 310 can be achromatized for the light beams within the wavelength range of the OCT light source 250. The optical lenses forming the OCT imaging module 310 from the distal end of the second imaging path, which includes the optical window 202, to the front of the optical fiber 103, which is the optical coupling lens 104, can be achromatized for the light with wavelengths used in the OCT imaging module 310. The optical lenses in the second imaging path can be further configured with additional consideration for the need to compensate the residual chromatic aberrations from the eye itself. Here the achromatization means the design to minimize the optical aberrations for multiple light wavelengths within the working wavelength range, not just one wavelength, as well as for the full field of view of the OCT imaging module 110. For example, the optical lenses in the second imaging path can be configured to minimize the optical aberrations at wavelengths of 1000 nm and 1080 nm for the working light wavelength range of 980 nm to 1100 nm, as well as for the field of view from 0 degree (on optical axis) to 130 degree.

In some other embodiments, the achromatization can be performed to minimize the axial color aberration for the selected light wavelengths for the full field of view of the OCT imaging module 310. The optical path difference for the light beams transmitting within the same portion of the optical lenses but with different optical wavelengths can be minimized to an allowable amount, and the same allowable amount of optical path difference can be applied to light beams transmitting through different portions of the optical lenses. For example, the light path difference for the light beams with optical wavelengths of 1000 nm and 1090 nm can be minimized to 2 nm for light beams transmitting along the optical axis and minimized to around 2 nm for light beam with the field of view of 130 degrees.

As discussed above, the optical lenses in the first imaging path of the first imaging module 120 can be achromatized for the light beams within the wavelength range of the first light source 221. The optical lenses shared by both the first imaging module 120 and the OCT imaging module 310, for example, the optical contact window 202, the imaging lens 204 and relay lens 205, can be achromatized for both optical wavelength bands in the first imaging module 120 and the OCT imaging module 310.

FIG. 4 is a schematic of an example OCT interferometer 280 of the wide field of view OCT imaging system 100 shown in FIG. 1. The OCT imaging system 100 can comprise the handheld imaging probe 140 and the console 130. The console 130 can comprise the OCT engine 131 and the scanning mirror control (not shown). The OCT engine 131 can comprise the OCT interferometer 280, the OCT light source 250, an optical detector 266, a data acquisition module 267, a processor 268, and a display. For example, the OCT interferometer 288 can be a fiber optical interferometer. The OCT light source 250 can comprise a swept light source in some embodiments. The OCT interferometer 280 can be the core of the OCT engine 131. The OCT interferometer 280 can work with different handheld imaging probes with different OCT imaging modules. For example, the OCT interferometer 280 can work with the OCT imaging module 110 in FIG. 2A, the OCT imaging module 310 in FIG. 3, and a variety of other OCT imaging modules.

In some embodiments, the light beam from the swept light source 250, which is linearly polarized, is guided through an optical fiber 251 to an optical fiber combiner 252. A light beam from an aiming light source 253 is also guided to the combiner 252. The light beam from the swept light source 250 is coupled with the light beam from the aiming light source 253 at the combiner 252. The output light beam from the combiner 252 is guided by a fiber 288. For example, the fiber 288 can be a polarization maintaining (PM) fiber 288. The fiber 288 can guide output light beam from the combiner 252 into a port of PM fiber optic coupler 255 with polarization of the light beam aligned with one of polarization axes of fiber 288 (slow axis for example), and then split into two light beams with various intensity ratios, (for example, ratios of 10:90, 20:80, or 30:70, or other ratios), which are then guided out through two PM optical fibers 256 and 257. A first portion of the output beam of the fiber optical coupler 255 can be guided to the PM fiber 256 to form a sample arm of the OCT interferometer 280, and a first portion of the output beam of the fiber optical coupler 255 can be guided to the PM fiber 257 to form a reference arm of the OCT interferometer 280. For example, a portion of the PM fiber 256 can be made into multiple small circles (turns) with small radius, as shown as 254 in FIG. 4A. Such treatment can be used to remove the residual light in one of axes of PM fiber 256, for example fast axis, making the light transmitting in the PM fiber 256 with only one linear polarization.

Referring to FIG. 1, FIG. 2A, FIG. 3, and FIG. 4, the light in the PM fiber 256 can be then further guided to the OCT imaging module 110 to form a sample arm of the optical fiber interferometer 280 in some embodiments. In some embodiments, the details of the sample arm are shown in FIG. 2A. In some other embodiments, the light beam in the PM fiber 256 can be further guided to the OCT imaging module 310 to form a sample arm of the optical fiber interferometer 280, while the sample arm comprising the OCT imaging module 310 as shown in FIG. 3. In some alternative embodiments, the light beam in the PM fiber 256 can be further guided to other OCT imaging modules to form a sample arm of the optical fiber interferometer 280. The PM fiber 256 can be connected to the handheld imaging probe 140 and becomes a portion of the optical fiber 103, which is shown in FIG. 1, FIG. 2A and FIG. 3. In some embodiments, the fiber 288 and the fiber 256 can form the fiber 103. The fiber 103 can comprise a first portion 288 and a second portion 256. In some other embodiments, the fiber 251, the fiber 288 and the fiber 256 can form the fiber 103. The fiber 103 can comprise a first portion 251, a second portion 288, and a third portion 256.

The light beam in the PM fiber 257 can be used to form a reference arm of the OCT interferometer 280, and is guided to an optical dispersion compensation module 258. The light beam from the PM fiber 257 is collimated by a collimator lens 259 and then coupled into another PM optical fiber 265 by a similar optical lens 260. The distance between the two optical lenses 259 and 260 can be changed to adjust the overall optical path length of the reference arm. The optical dispersion component 261 can comprise various optical materials matching optical dispersion properties of the optical materials used to make the optics in the sample arm. The optical dispersion component 261 can also include an optical filter which absorbs or reflects the light beam from the aiming light source 253 and prevent the aiming light beam from going through the dispersion compensation module 258.

Referring to FIG. 2A and FIG. 4, the light beam scattered form the posterior segment, the retina, of the eye can be received by the OCT imaging module 110 and sent back through the optical fiber 256. The light beam is then returned to the fiber optic interferometer 280 through the PM optical fiber 256. Although the returned light beam comprising light beams originated from the swept light source 250 and the aiming light source 253, the intensity of the aiming light is more attenuated because the fact that the beam splitting dichroic mirror 102 within the OCT Imaging Module 110 transmits a significant portion of the light beam of the aiming light source 253 to the image sensor 201.

A portion of the light beam from the PM fiber 256 can be split by the optical fiber coupler 255 again, which is then coupled to another PM fiber 262. A polarization adjustment component 263 can be used to adjust the polarization direction of the light transmitting from the fiber 262 to another 50:50 optical fiber coupler 264, and to align with the polarization direction of light beam from the fiber 265 in the reference arm. The light beams from the sample arm fiber 262 and the reference arm fiber 265 can be mixed in the fiber coupler 254, and results in two interfering light beams with a constant 180 degree shift in their optical phase difference, which are then coupled out in two optical fibers. The balance optical detector 266 can be used to detect the light signals from the two fibers and converts them into electrical signal in the form of differential detection. The digital acquisition module 267 can be used to digitize the analog signal which is then sent to the processor 268 for further processing and for generating OCT images for physicians to make medical diagnosis. The processor 268 is configured to process data from the interferometer and to generate the OCT image of the eye from the OCT imaging module. Because most of the light beam from the aiming light source 253 is removed before reaching the balance detector 266, it cannot cause any significant effect in the detected signals. In some other embodiments, the balance detector 266 can be configured such that the detector 266 is not sensitive to the light wavelength range of the aiming light source 253.

For conventional OCT imaging systems where the optical fibers between the OCT engine and the scanning probe are relative short and fixed in space, the motion effect, such as bending or twisting of the fibers, may not be significant on those fibers during the operation of OCT imaging. In general, conventional OCT imaging systems do not include PM fibers. However, in order to be connected to a portable handheld imaging probe, the fibers can be long, for example, as long as 5 meters. For the portable handheld OCT imaging probe, because the fibers are long and being part of the optical interferometer, such motion effect on the fibers, for example, bending or twisting of fibers, can introduced unwanted instability of the OCT images and affect the image quality significantly. One of the most significant problems for the conventional handheld OCT imaging probes is such motion effect.

As shown in FIG. 4, the use of polarization fibers (e.g., 256), and the related optical design for the OCT engine 131 can reduce and minimize such motion effect. Because the special prosperity of PM fibers (e.g., 256) and the special treatment to remove the light in one of axis entirely, the external effects, such as motion effect from bending or twisting of the PM fibers 256 and 257, cannot change the polarization direction of light beam transmitting in the fibers. Therefore, the resulted OCT images are more stable. The stabilization of the polarization direction for the light beam in the sample arm is very important in stabilizing the optical interference pattern in the fiber interferometer 280, which can result in more stable OCT images and increase the image quality significantly.

The use of optical polarization fibers (e.g., 256) may result in high optical signal loss. Because FDA regulations, only limited amount of light can be projected into the eye for safety concern. The amount of light returns from the eye is very small for ophthalmic OCT imaging systems. The potential high loss in the fibers can reduce the signal-to-noise ratio of the OCT images. Considerations need to be taken into in the related OCT imaging module design in order to increase the signal-to-noise ratio of the OCT images.

The OCT interferometer 280 can comprise one or more PM fibers to reduce motion effect of the fibers in order to increase the stability of optical quality of the OCT images. In some embodiments, the OCT interferometer 280 can comprise a plurality of PM fibers, for example, fiber 256 and fiber 257 can be PM fibers. In some alternative embodiments, the OCT interferometer 280 can comprise one PM fiber 256, where other fibers are regular, non PM fibers.

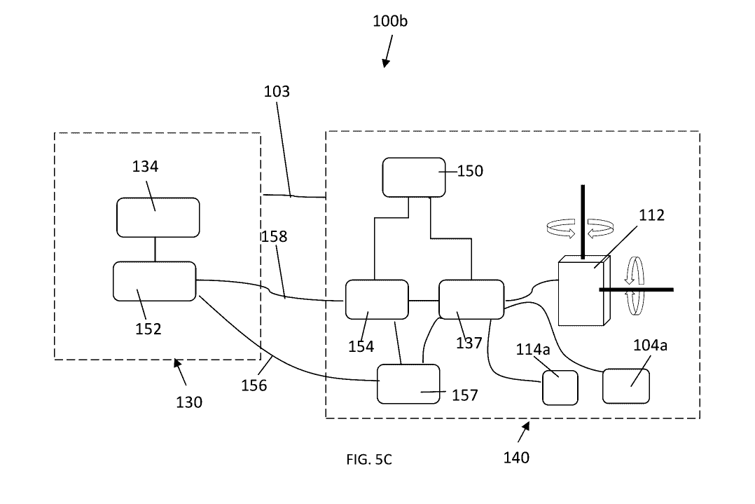

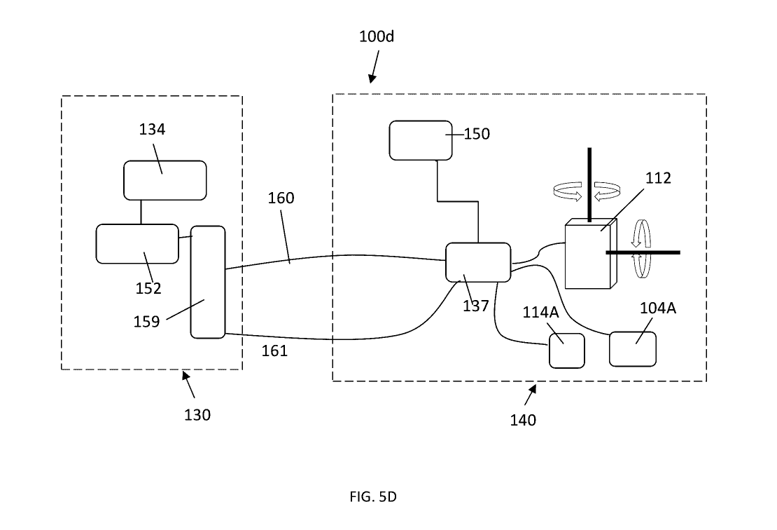

FIG. 5A schematically illustrates a wide field of view OCT imaging system 100a with an electrical cable 136a as a date link to connect the console 130 and the handheld imaging probe 140. Referring to FIG. 1, FIG. 2A and FIG. 5A, the OCT imaging system 100 can further comprise an electric driver 137. The electric driver 137 can be housed in the OCT Imaging Module 110 within the Imaging Probe 140, which controls and powers up the scanning mirror 112 which can in either one direction or two orthogonal directions. The electric driver 137 also can be used to control an actuator 104a for the lens 104 and an actuator 114a for the focusing lens 114. The corresponding scanning mirror controller 134 for the electric driver 137, which can be housed in the main console 130, communicates and synchronizes the motions through the data link. In one embodiment, the data link is provided by the electric cable 136a The synchronization of the scanning mirror 112, as well as the actuators 114a and 104a, with the OCT light source (not shown) and the data acquisition (not shown) can be carried out through the electric cable 136a. The electric cable 136a can connect the scanning mirror controller 134 in the console 130 to the scanning MEMS mirror 112 in the probe 140. The electrical cable 136 also provides electric power to the OCT Imaging Module 110 in the probe 140. The communication between the console 130 and Imaging Probe 140 for other imaging modalities can be carried out with other connections as well.

FIG. 5B schematically illustrates a wide field of view OCT imaging system 100b with an integrated OCT imaging module 110 comprising at least one OCT fibers 103 and a second optical synchronization fiber (or fibers) 158. For the remote imaging probe 140, which can come into contact with patient directly or through a disposable cap, the electrical isolation from the console 130 becomes a major issue for the electrical safety; especially when the high voltage is involved with the scanning mirror 112 itself. For example, high voltages can be applied to a MEMS scanning mirror 112 in some embodiments. In order to provide necessary electrical isolation, the connection between the scanning mirror controller 134 and the OCT Imaging Module 110 can be carried out by the second optical synchronization fiber (or fibers) 158 in some embodiments. The OCT fibers 103 and the second synchronization optical fiber(s) 158 can be connected to the console 130 and the imaging probe 140.

FIG. 5C further illustrates the details of the OCT imaging system 100b comprising the second synchronization optical fiber(s) 158. The signal from the controller 134 can be first sent to an electric/optical converter 152 which converts the electric signal into an optical synchronization signal, then sent through the second optical fiber(s) 158, either single mode or multimode optical fiber(s), to the Imaging Probe 140. The optical signal can be received by an optic/electrical converter 154 which converts the signal into the electric signal and used to synchronize the electric driver 137 housed in the OCT Imaging Module 110. The communication can be both ways. In some embodiments, the optic/electrical converter 154 and electric driver 137 can be both powered by an internal battery 150 housed in the Imaging probe 140. The electrical/optical converter 152 not only converts electrical signal into electrical signal, it also can convert electrical power into optical power through a relatively high power module. The optical power is then transmitted through another multiple mode fiber 156 to the Imaging Probe 140. The light from the fiber 156 is then received by an optical/electrical power convertor 157 and converts the optical power into electrical power. The electrical power from the convertor 157 is then used to power optical/electrical signal convertor 154 and the electric driver 137. Through such design, the total electrical isolation between the console 130 and Imaging Probe 140 is realized. It should be pointed out that the optical signal can also be carried out by the OCT fiber(s) 103 in some other embodiments.

FIG. 5D schematically illustrates another embodiment for a wide field of view OCT imaging system 100d, where the electrical isolation between the console 130 and Imaging Probe 140 is implemented. An electrical isolator 159 which provides electrical isolation for both electrical signal and electrical power through optical or electromagnetic methods. After the electrical signal from the electric controller 134 goes through the electrical isolator 159, the signal remains the same, but electrically isolated from the electric ground of the console 130 and is then transmitted to Imaging Probe 140 through an electrical wire 160. The signal is then used to synchronize the electric controller 134 and the electric driver 137. The electrical isolator 159 also provides electric power which is electrically isolated from the electric ground of console 130, to the electric driver 137 through the electric wire 161.

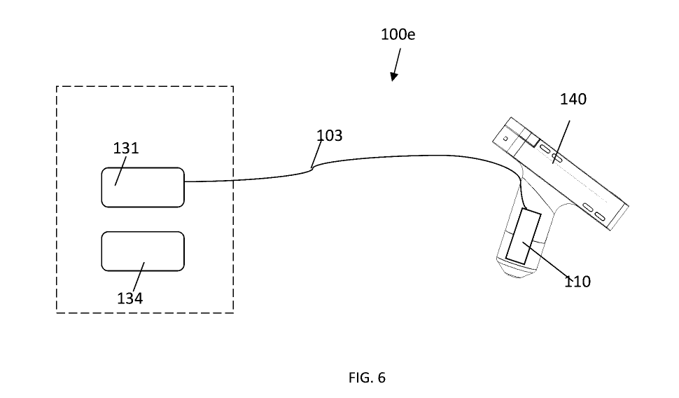

FIG. 6 schematically illustrates another embodiment for a wide field of view OCT imaging system 100e, where the synchronization between the scanning mirror controller 134 and the driver 137 is realized wirelessly, thus eliminating the need for either the electric cable or the optical fiber for communication purpose. The wireless transmission can be in the form of WiFi, Bluetooth, or other special wireless communication protocols.

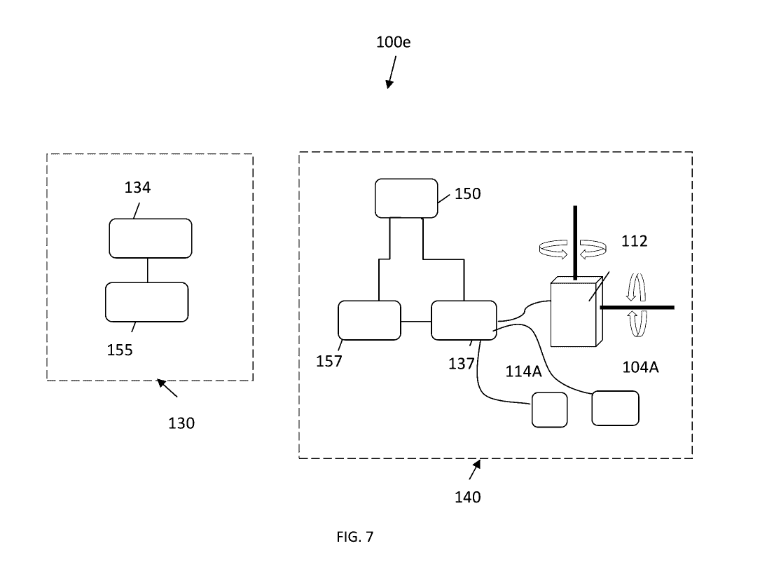

FIG. 7 schematically illustrates the details of the synchronization between the broadband light source, the data acquisition system and the scanning MEMS mirror wirelessly. As shown in the FIG. 7, the signal from the scanning mirror controller 134 can be first sent to a first wireless transponder 155, which converts and sends out the signal wirelessly. The corresponding second wireless transponder 157, housed in the Imaging Probe 140, can convert the signal into electric signal and sent to the scanning mirror driver 137 to control the motion of the scanning mirror 112 and focusing mechanisms. The power for such wireless transponders 157 and electric driver 137 can be supplied by the internal battery 150 housed in the Imaging Probe 140 in some embodiments. The wireless communication can be in both ways.

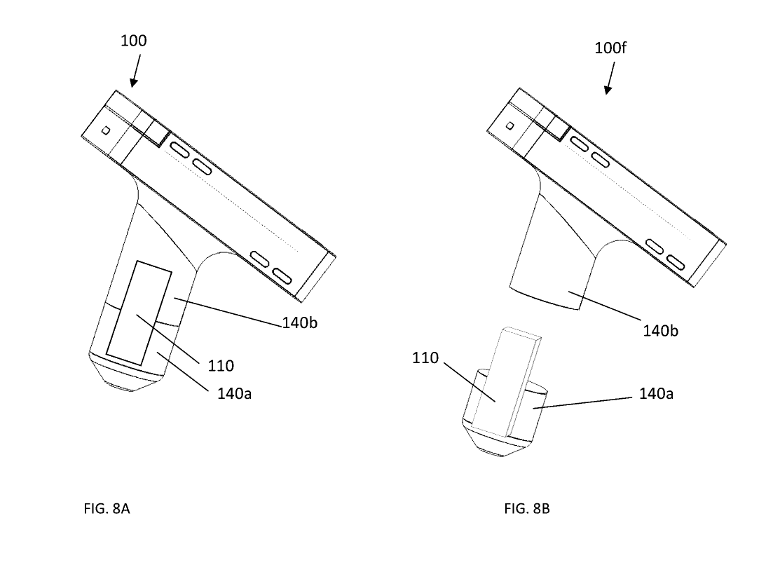

FIG. 8A schematically illustrates that the wide field of view OCT system 100 with the OCT imaging module 110 implemented as a fully integrated part of the handheld imaging probe 140. FIG. 8B schematically illustrates that a wide field of view OCT system 100f where the OCT imaging module 110 can be implemented as a detachable module 140a attached to the main portion 140b of the imaging probe 140. The imaging probe 140 may comprise the removable front imaging module 140a and the main module 140b. The imaging probe 140 may be built as one piece or two separate pieces, as shown as 140a and 140b, in the FIG. 8A and FIG. 8B. The length of the front portion 140a can be from 30 mm to 50 mm. The OCT imaging module 110 can be disposed on the housing of the front removable section 140a of the imaging probe 140. In some embodiments, the front imaging module 140a may be removed or replaced with other functioning modules which may contain different optics. For example, front imaging modules with higher magnification, front imaging modules designed for premature babies, front imaging modules designed for adult, front imaging modules designed for fluorescein angiography imaging, front imaging modules for NIR imaging and front imaging modules for anterior segment imaging can be used in different circumstances. Accordingly, in designs where the front imaging module is replaceable or removable, the potential use or applications of the imaging probe 140 may be significantly expanded.

In some embodiments, the Imaging Probe 140 may be used to acquire images of the posterior segment of the eye with various magnifications and under the illumination from broadband or narrow spectral light sources. The spectrum of the light source may be in the visible, IR, near IR, UV light range or combinations thereof. To obtain a wide field of the view (FOV), the optical window may be placed over the cornea of the eye directly or through a disposable cap with slight pressure. Accordingly, the optical window may have a concave surface matching the size of the cornea. In some embodiments, for example, the outer surface of the optical window has a radius of curvature of between 6 mm and 15 mm. An optical transparent index matching gel with sufficient viscosity may be placed between the cornea and the optical window. The viscosity of the index matching gel may be at least 100 centipoise, 200 centipoise or 300 centipoise. The iris of the patient may or may not be dilated with special drugs. In some embodiments, the Imaging Probe 140 may also be used to obtain images of the anterior segment of the eye by using a front imaging module 140a designed for imaging the anterior segment, using the same illumination system.

In FIG. 8B, different types of imaging modules can be used by the operator to perform different imaging modalities, with or without the OCT imaging capability included, thus expanding the capability of the handheld imaging probe 140. The attachment can be carried out through a common/standard mechanical locking/interface, which not only provide secured mechanical connection, but also provide connection for electrical power and electrical signal synchronizations. The OCT Imaging Module 110 can be implemented with other imaging modalities to form different types of imaging format, for example, OCT with color imaging, OCT with Fluorescing angiography imaging, OCT with auto-fluorescence imaging. The OCT Imaging Module 110 can also be implement with different imaging optics with different fields of view, for example, wide-field imaging or high magnification imaging.

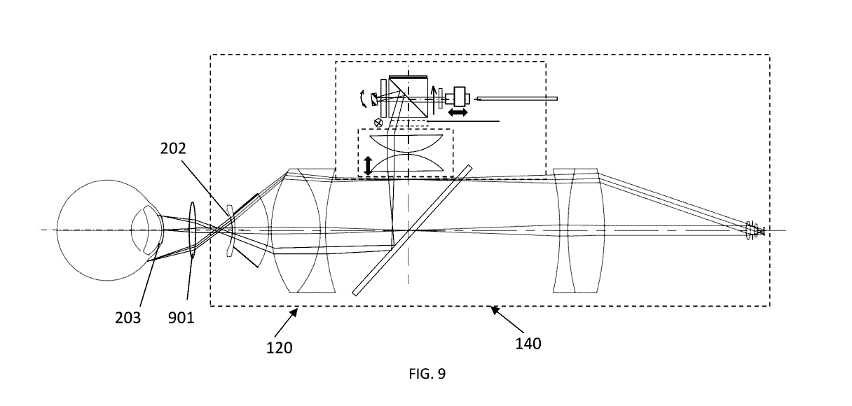

FIG. 9 schematically illustrates the same OCT Imaging Module 110 can be used to image an anterior segment 203 of the eye in one embodiment of the disclosure. The OCT Imaging Module 110 can also be used to image the anterior segment 203, the cornea, of the eye when the optical window is moved away from and kept at a distance from the cornea 203 of the eye, with or without additional optics attached to the front of the optical window 202, in the form of non-contact microscope. The first imaging module 120 can provide a real-time 2D color image of the anterior segment 203 of the eye with tracks for the location of the OCT scanning beam while the OCT imaging module 110 can provide a real-time sectional view of the same region. The user can select the direction of the scanning for the sectional view and location of the sectional view. If the target is stable enough, a full field 3D OCT image can be taken with a still color image. In some other embodiments, an optional adaptor lens 901 can be added to the front of the Imaging Probe 140 to improve the effect of the image distortion for the anterior imaging. The first imaging module 120 can have the same field of view as the OCT imaging module 110.

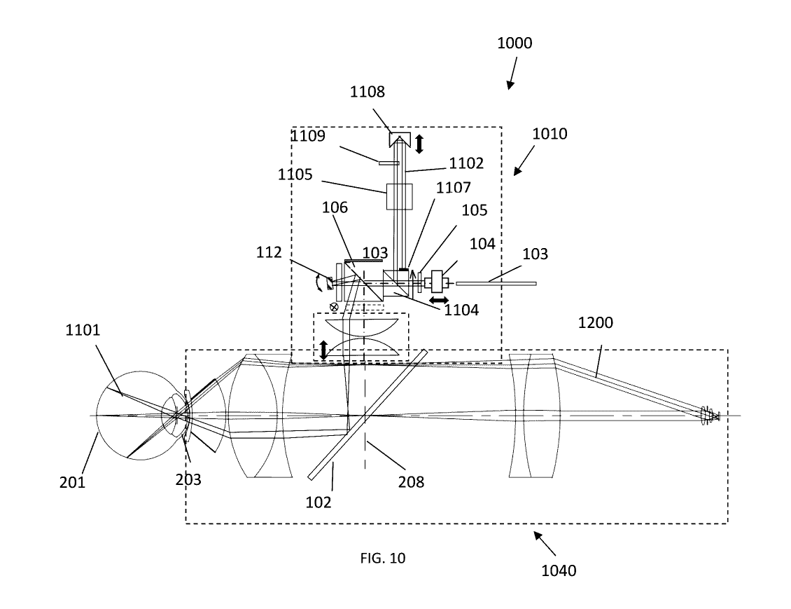

FIG. 10 is a schematic of an example optical design of a wide field of view OCT imaging system 1000, where a reference arm 1102 of an OCT interferometer is integrated into a handheld OCT imaging probe 1040 according to another embodiment of the disclosure. The Imaging Probe 1040 of the OCT imaging system 1000 can comprise the OCT Imaging Module 1010 integrated with the reference arm 1102 for the OCT interferometer. In some embodiments, the OCT sample arm 1101 and the reference arm 1102 can be configured to share the light beam transmitted from the optical fiber 103. The sharing of the fiber 103 can help to minimize the drifting of the OCT images and stabilize from undesired movement for the OCT images, which is introduced by the motion effect of the optical fiber 103, such as bending or twisting. In some embodiments, the fiber 103 can be a polarization maintaining fiber, which can be used to transmit the light in the one of the polarization axis, for example slow axis, of the fiber 103 only.

After the light from the polarizing maintaining fiber 103 is collimated by the fiber coupling lens 104 and passes through the linear polarizer 105, the light beam can be split by a beam splitter 1104 into the sample beam 1101 and the reference beam 1102, for example, at a ratio of 20/80, 10/90, etc. The sample beam 1101 can continue to propagate to the polarizing beam splitter 106, then to the scanning mirror 112; while the reference beam 1102 can be projected into a relay path to provide an equal optical length as the sample beam 1101.

In some embodiments, an optical dispersion correction module 1105 can be used to compensate the wavelength depended dispersion and polarization dependent dispersion in the reference beam 1102 in the relay path. The wavelength depended dispersion and polarization dependent dispersion can occur in the sample optical path 1101 due to the existence of optical dispersion in the optical materials used in the imaging optics and biological tissues in the eye. A retro-reflector 1108 and a reflective mirror or reflective coating 1107 which is added to surface of the beam splitter 1104 can be used to reflect the reference light beam 1102 and form a compact relay path. After multiple reflections, the light as reference beam can be reflected by the beam splitter 1104 and back to the linear polarizer 105 and polarization maintaining fiber 103 in the same optical axis, for example, the slow axis. A light intensity modulator 1109 can also inserted into the reference light beam 1102 to control its brightness, thus provides the adjustment for the overall light power of the optical interference signal which is used to generate the OCT images when the OCT Imaging Module 110 is used to image different objects. The light intensity modulator 1109 can be constructed in the form of a broadband variable neutral density filter, a broadband half-wave plate, a linear polarizer, and a liquid crystal modulator. The retro-reflector 1108 can be configured to be movable along the optical axis of the reference beam 1102, which provides adjustment for the optical path in the reference beam 1102 such that the sample path length and reference path length are nearly equal. The working distance range of the OCT imaging module 110 can be adjusted by moving the retro-reflector 1108 based on pre-calibrated distance, when the operator switches from performing OCT imaging of the posterior segment to the anterior segment of the eye, as demonstrated in FIG. 9. The optical dispersion correction module 1105 can also be removed or adjusted to better match with the optical dispersion in the sample beam 1101 when the anterior segment and the posterior segment OCT imaging are performed.

In general, disclosed herein is a wide field of view OCT imaging system for a variety of applications, not being limited to ophthalmic application. For example, the OCT imaging system can be used to image an ear, a nose or other body parts of human, or a variety of other samples. The optical window can be configured to be in contact with the sample and the front surface of the optical window can be configured to match a shape of the sample. For example, the OCT imaging system can be designed to obtain an OCT image of an ear, and the optical window can be configured to have a front surface matching the shape of the ear drum.

In some embodiments, the OCT imaging system for viewing a sample can comprise a handheld imaging probe and a console. The handheld imaging probe can comprise a probe housing with a distal end, a first imaging module and an OCT imaging module. The first imaging module can be disposed on a main portion of the probe housing. The OCT imaging module can be disposed on a side portion of the probe housing. The first imaging module can comprise a first illumination path and a first imaging path, where the first imaging path is separated from the first illumination path. The first illumination path comprises a first light source disposed inside the probe housing and a light conditioning element having multiple segments. The light conditioning element is positioned behind a peripheral portion of an optical window. The light conditioning element is configured to receive a first light beam from the first light source and directionally control the first light beam to the sample. The first imaging path can comprise an optical window disposed at the distal end of the probe housing and configured to be in contact with the sample. The optical window can have a front surface mating a shape of the sample to be viewed. The first imaging path can comprise one or more lenses, which include a first focusing lens to adjust a focus of the first imaging module. The first imaging path can further comprise an image sensor configured to receive a first image of the sample. The OCT imaging module can comprise a second illumination path and a second imaging path. The second illumination path and the second imaging path can comprise a scanning MEMS mirror and a beam splitting dichroic mirror. The scanning MEMS mirror is configured to scan a first portion of an OCT light beam from an OCT light source. The scanning MEMS mirror can be disposed outside the first illumination path and the first imaging path. The beam splitting dichroic mirror can be disposed in the first imaging path and configured to transmit the first light beam and reflect the first portion of the OCT light beam. The second illumination path and the second imaging path can further comprise a second focusing lens to adjust a focus of the OCT imaging module.

In some embodiments, a wide field of view OCT system can comprise a handheld imaging probe. The handheld imaging probe can have an OCT imaging module. The handheld imaging probe can comprise a probe housing with a distal end and an optical window disposed at the distal end. The optical window can have a front surface matching a shape of a sample and is configured to be in contact with the sample. The handheld imaging probe can further comprise a scanning MEMS mirror configured to scan a first portion of a light beam from a light source. The handheld imaging probe can further comprise one or more lenses in an imaging path. The wide field of view OCT system can comprise a console. The console can comprise the light source, an interferometer, a processor, a scanning MEMS mirror controller and a datalink. The interferometer can comprise at least one polarization maintaining fiber, the at least one polarization maintaining fiber can be configured to couple a light beam from the light source to the handheld imaging probe and reduce motion effect to stabilize and increase a quality of an OCT image. The processor can be configured to process data from the interferometer and to generate the OCT image of the sample from the OCT imaging module. The data link is connected to the console and the handheld imaging probe and configured to synchronize the scanning MEMS mirror with the light source and the scanning MEMS mirror controller.

In some embodiments, a wide field of view OCT system can comprise a handheld imaging probe. The handheld imaging probe can have an OCT imaging module. The handheld imaging probe can comprise a probe housing with a distal end and an optical window disposed at the distal end. The optical window can have a front surface matching a shape of a sample and is configured to be in contact with the sample. The handheld imaging probe can further comprise a scanning MEMS mirror configured to scan a first portion of a light beam from a light source. The handheld imaging probe can further comprise one or more lenses in an imaging path, where the one or more lenses are achromatized for optical dispersion for the light beams within a wavelength range of the light source and for a field of view of the OCT imaging module. The OCT system can comprise a console. The console can comprise the light source, an interferometer, a processor, a scanning MEMS mirror controller and a datalink. The processor can be configured to process data from the interferometer and to generate the OCT image of the sample from the OCT imaging module. The data link is connected to the console and the handheld imaging probe and configured to synchronize the scanning MEMS mirror with the light source and the scanning MEMS mirror controller.

While the present disclosure has been disclosed in example embodiments, those of ordinary skill in the art will recognize and appreciate that many additions, deletions and modifications to the disclosed embodiments and their variations may be implemented without departing from the scope of the disclosure. A wide range of variations to those implementations and embodiments described herein are possible. Components and/or features may be added, removed, rearranged, or combinations thereof. Similarly, method steps may be added, removed, and/or reordered.

Likewise various modifications to the implementations described in this disclosure may be readily apparent to those skilled in the art, and the generic principles defined herein may be applied to other implementations without departing from the spirit or scope of this disclosure. Thus, the claims are not intended to be limited to the implementations shown herein, but are to be accorded the widest scope consistent with this disclosure, the principles and the novel features disclosed herein.

Accordingly, reference herein to a singular item includes the possibility that a plurality of the same item may be present. More specifically, as used herein and in the appended claims, the singular forms “a,” “an,” “said,” and “the” include plural referents unless specifically stated otherwise. In other words, use of the articles allow for “at least one” of the subject item in the description above as well as the claims below.

Additionally as used herein, a phrase referring to “at least one of” a list of items refers to any combination of those items, including single members. As an example, “at least one of: a, b, or c” is intended to cover: a, b, c, a-b, a-c, b-c, and a-b-c.

Certain features that are described in this specification in the context of separate embodiments also can be implemented in combination in a single embodiment. Conversely, various features that are described in the context of a single embodiment also can be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations may be described as occurring in a particular order, this should not be understood as requiring that such operations be performed in the particular order described or in sequential order, or that all described operations be performed, to achieve desirable results. Further, other operations that are not disclosed can be incorporated in the processes that are described herein. For example, one or more additional operations can be performed before, after, simultaneously, or between any of the disclosed operations. In certain circumstances, multitasking and parallel processing may be advantageous. Moreover, the separation of various system components in the embodiments described above should not be understood as requiring such separation in all embodiments, and it should be understood that the described program components and systems can generally be integrated together in a single product or packaged into multiple products. Additionally, other embodiments are within the scope of the following claims. In some cases, the actions recited in the claims can be performed in a different order and still achieve desirable results.

The systems, devices, and methods of the preferred embodiments and variations thereof can be embodied and/or implemented at least in part as a machine configured to receive a computer-readable medium storing computer-readable instructions. The instructions are preferably executed by computer-executable components preferably integrated with the system including the computing device configured with software. The computer-readable medium can be stored on any suitable computer-readable media such as RAMs, ROMs, flash memory, EEPROMs, optical devices (e.g., CD or DVD), hard drives, floppy drives, or any suitable device. The computer-executable component is preferably a general or application-specific processor, but any suitable dedicated hardware or hardware/firmware combination can alternatively or additionally execute the instructions.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical range recited herein is intended to include all sub-ranges subsumed therein.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived therefrom, such that structural and logical substitutions and changes may be made without departing from the scope of this disclosure. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A wide field of view optical coherence tomography (OCT) imaging system comprising:
a handheld imaging probe comprising:
a probe housing with a distal end;
a first imaging module disposed on a main portion of the probe housing, the first imaging module comprising:
a first illumination path comprising
a first light source disposed inside the probe housing;
a light conditioning element having multiple segments and positioned behind a peripheral portion of an optical window, the light conditioning element configured to receive a first light beam from the first light source and directionally control the first light beam to an eye; and
a first imaging path separated from the first illumination path, the first imaging path comprising
an optical window disposed at the distal end and configured to be in contact with a cornea of the eye, the optical window having a concave front surface;
a first focusing lens to adjust a focus of the first imaging module; and
an image sensor configured to receive a first image of the eye; and
an OCT imaging module disposed on a side portion of the probe housing, the OCT imaging module comprising:
a second illumination path and a second imaging path, the second illumination path and the second imaging path comprising
a scanning MEMS mirror configured to scan a first portion of an OCT light beam from an OCT light source, the scanning MEMS mirror disposed outside the first illumination path and the first imaging path;
a beam splitting dichroic mirror disposed in the first imaging path and configured to transmit the first light beam and reflect the first portion of the OCT light beam; and
a second focusing lens to adjust a focus of the OCT imaging module.

2. The OCT imaging system of claim 1, wherein the scanning MEMS mirror is positioned in an optical conjugate plane of an entrance pupil of the first imaging system.

3. The OCT imaging system of claim 1, wherein a real image of an aperture of the OCT imaging module is positioned near an anterior surface of the crystalline lens of the eye when a posterior segment of the eye is imaged.

4. The OCT imaging system of claim 1, further comprising an imaging lens and a first relay lens disposed in the first imaging path and optically aligned with the optical window, wherein the beam splitting dichroic mirror is disposed behind the first relay lens, and wherein the second imaging path and the first imaging path share optical components only until the first relay lens.

5. The OCT imaging system of claim 1, wherein a field of view of the OCT imaging module is at least 120 degrees×120 degrees but no more than 180 degrees×180 degrees in a single volume acquisition.

6. The OCT imaging system of claim 1, wherein the first light source has a first wavelength range between 450 nm to 700 nm, inclusive.

7. The OCT imaging system of claim 1, wherein the first light source has a first wavelength range from 700 nm to 840 nm in the near infrared light range.

8. The OCT imaging system of claim 1, wherein the optical window and the OCT imaging module are disposed on a removable front imaging module of the handheld imaging probe, wherein the removable front imaging module are configured to be repeatedly detached from, and re-attached to, the handheld imaging probe without using tools.

9. The OCT imaging system of claim 1, wherein the first focusing lens is achromatized for optical dispersion for the first light beam within a wavelength range of the first light source and for a field of view of the first imaging module.

10. The OCT imaging system of claim 1, wherein the second focusing lens is achromatized for optical dispersion for the OCT light beam within a wavelength range of the OCT light source and for a field of view of the OCT imaging module.

11. The OCT imaging system of claim 1, further comprising a console, wherein the console comprises the OCT light source, an interferometer, and a first optical fiber, wherein the first optical fiber is connected to the console and the handheld imaging probe and configured to couple the first portion of the OCT light beam from the OCT light source to the handheld imaging probe to form a sample arm of the interferometer.

12. The OCT imaging system in claim 11, wherein the OCT light source is a swept-source laser.

13. The OCT imaging system of claim 11, wherein the console further comprises a second optical fiber configured to couple the second portion of the OCT light beam to a reference arm of the interferometer, the reference arm disposed in the console.

14. The OCT imaging system of claim 11, wherein the handheld imaging probe further comprises a reference arm of the OCT interferometer, wherein the second portion of the OCT light beam is coupled to the reference arm by the first optical fiber.

15. The OCT imaging system of claim 11, further comprising a processor configured to process data from the interferometer and to generate an OCT image of the eye from the OCT imaging module.

16. The OCT imaging system of claim 15, wherein the handheld imaging probe further comprises a wireless transmitter, a wireless receiver and a display, the display configured to present the first image and the OCT image simultaneously.

17. The OCT imaging system of claim 15, wherein the first optical fiber is configured to maintain a polarization of the OCT light beam to reduce motion effect of the first optical fiber to stabilize and increase the OCT image quality.

18. The OCT imaging system of claim 17, wherein the first optical fiber comprises a plurality of turns to remove the residual light beam in one of axes of the optical fiber such that the light beam transmitting in the first with only one linear polarization.

19. The OCT imaging system in claim 15, comprising a scanning MEMS mirror controller disposed in the console and an electrical cable connecting the console to the handheld imaging probe, wherein the scanning mirror controller and the scanning MEMS mirror are synchronized through the electrical cable.

20. The OCT imaging system in claim 15, comprising a scanning MEMS mirror controller disposed in the console, an electrical-optical converter disposed in the console and configured to convert an electrical signal into an optical signal, an optical-electrical converter disposed in the handheld imaging probe and configured to convert the optical signal back into the electrical signal, and a third optical fiber connected to the console to the handheld imaging probe, the third optical fiber is configured to transmit the optical signal, wherein the scanning mirror controller, the scanning mirror driver and the scanning MEMS mirror are synchronized through the third optical fiber.

21. The OCT imaging system in claim 15, comprising a pair of wireless transponders, one wireless transponder disposed in the console and the other wireless transponder disposed in the handheld imaging probe, wherein the scanning mirror controller and the scanning MEMS mirror are synchronized wirelessly via the wireless transponders.

22. The OCT imaging system of claim 15, further comprising a third light source disposed in the console and a beam combiner disposed in the console, the beam combiner is configured to couple both the OCT light source and the third light source to the handheld imaging probe through the first optical fiber, the third light source having a third light beam, the third light beam having a third illumination path and a third imaging path, wherein the third illumination path is along the second illumination path of the OCT imaging module and the third imaging path is along the first imaging path of the first imaging module, wherein the beam splitting dichroic mirror is configured to partially reflect and partially transmit the third light beam.

23. The OCT imaging system of claim 22, wherein a track of the third light beam is configured to provide registry of an imaging location of the OCT light beam and provide a feedback to control the second focusing lens, wherein a first adjustment of the first focusing lens and a second adjustment of the second focusing lens are synchronized through the feedback.

24. A wide field of view optical coherence tomography (OCT) imaging system comprising:
a handheld imaging probe having an OCT imaging module, the handheld imaging probe comprising:
a probe housing with a distal end;
an optical window disposed at the distal end and configured to be in contact with a cornea of an eye, the optical window having a concave front surface;
a scanning MEMS mirror configured to scan a first portion of a light beam from a light source;
one or more lenses in an imaging path; and
a console comprising:
the light source,
an interferometer comprising at least one polarization maintaining fiber, the at least one polarization maintaining fiber configured to couple a light beam from the light source to the handheld imaging probe and reduce motion effect to stabilize and increase a quality of an OCT image of the eye,
a processor configured to process data from the interferometer and to generate the OCT image from the OCT imaging module
a scanning MEMS mirror controller; and
a data link connecting the console to the handheld imaging probe and configured to synchronize the scanning MEMS mirror with the light source and the scanning MEMS mirror controller.

25. The OCT imaging system of claim 24, wherein a field-of view of the OCT imaging module is at least 120 degrees×120 degrees but no more than 180 degrees×180 degrees in a single volume acquisition.

26. The OCT imaging system of claim 24, wherein the one or more lenses are achromatized for optical dispersion for the light beams within a wavelength range of the light source.

27. The OCT imaging system of claim 24, wherein the one or more lenses are achromatized for optical dispersion for a field of view of the OCT imaging module.

28. The OCT imaging system in claim 24, wherein the light source is a swept-source laser.

29. The OCT imaging system of claim 24, wherein the at least one polarization maintaining fiber comprises a plurality of turns to remove the residual OCT light beam in one of axes of the polarization maintaining fiber such that the light beam transmitting with only one linear polarization.

30. The OCT imaging system in claim 24, wherein the data link is an electrical cable.

31. The OCT imaging system in claim 24, wherein the data link is a second optical fiber cable.

32. The OCT imaging system in claim 24, wherein the data link is wireless.

33. The OCT imaging system in claim 24, further comprising a wireless transmitter, a wireless receiver and a display, the display configured to present the OCT image.

34. A wide field of view optical coherence tomography (OCT) imaging system comprising:
a handheld imaging probe having an OCT imaging module, the handheld imaging probe comprising:
a probe housing with a distal end;
an optical window disposed at the distal end and configured to be in contact with a cornea of an eye, the optical window having a concave front surface;
a scanning MEMS mirror configured to scan a first portion of a light beam from a light source;
one or more lenses in an imaging path, the one or more lenses are achromatized for optical dispersion for the light beams within a wavelength range of the light source and for a field of view of the OCT imaging module; and
a console comprising:
the light source,
an interferometer comprising a plurality of fibers,
a processor configured to process data from the interferometer and to generate the OCT image of the eye from the OCT imaging module;
a scanning MEMS mirror controller; and
a data link connecting the console to the handheld imaging probe and configured to synchronize the scanning MEMS mirror with the light source and the scanning MEMS mirror controller.

35. The OCT imaging system of claim 34, wherein a field-of view of the OCT imaging module is at least 120 degrees×120 degrees but no more than 180 degrees×180 degrees in a single volume acquisition.

36. The OCT imaging system in claim 34, wherein the light source is a swept-source laser.

37. The OCT imaging system in claim 34, wherein the data link is another optical fiber cable.

38. The OCT imaging system in claim 34, wherein the data link is wireless.

39. The OCT imaging system in claim 34, further comprising a wireless transmitter, a wireless receiver and a display, the display configured to present the OCT image.

40. A method of obtaining a wide field of view optical coherence tomography (OCT) image, the method comprising:
holding a handheld imaging probe in a hand,
placing an optical window disposed at a distal end of the handheld imaging probe in contact with a cornea of an eye;
illuminating the eye with a first light beam transmitted from a first light source through a light conditioning element positioned behind a peripheral portion of the optical window along a first illumination path;
directional controlling the first light beam with the light conditioning element;
obtaining a first image of the eye through a first imaging path, the first imaging path separated from the first illumination path;
scanning an OCT light beam from an OCT light source with a scanning MEMS mirror, the scanning MEMS mirror disposed outside the first illumination path and the first imaging path;
reflecting the first portion of the OCT light beam off of a beam splitting dichroic mirror to the eye, and receiving a reflected OCT light beam from the eye; and
obtaining the OCT image of the eye.

41. The method of claim 40, wherein obtaining a first image of the eye comprises obtaining a first image of the eye with one or more lenses achromatized for optical dispersion for the first light beam within a wavelength range of the first light source and for a field of view of the first image.

42. The method of claim 40, wherein obtaining the OCT image of the eye comprises obtaining the OCT image of the eye with one or more lenses achromatized for optical dispersion for the OCT light beam within a wavelength range of the OCT light source and for a field of view of the OCT image.

43. The method of claim 40, further comprising using a swept-source laser as the OCT light source.

44. The method of claim 40, further comprising presenting the first image and the OCT image simultaneously on a display on the handheld imaging probe.

45. The method of claim 40, further comprising coupling the OCT light beam from the OCT light source to the handheld imaging probe with a polarization maintaining optical fiber to reduce motion effect to stabilize and increase the OCT image quality.

45. The method of claim 40, further comprising providing registry of an imaging location of the OCT light beam with a third light beam from a third light source, the third light beam having a third illumination path along an illumination path of the OCT imaging module and a third imaging path along the first imaging path of the first imaging module.

46. The method of claim 45, further comprising synchronizing a first focus adjustment of the first image and a second focus adjustment of the OCT image with a feedback from a track of the third light beam.

47. A method of obtaining a wide field of view optical coherence tomography (OCT) image, the method comprising:
holding a handheld imaging probe in a hand,
placing an optical window disposed at a distal end of the imaging probe in contact with a cornea of an eye;
illuminating the eye with an OCT light beam from an OCT light source, the OCT light source being coupled to the handheld imaging probe by a polarization maintaining fiber to reduce motion effect and to increase OCT image quality;
scanning a first portion of the OCT light beam by using a scanning MEMS mirror; and
obtaining the OCT image of the eye.

48. The method of claim 47, wherein obtaining the OCT image of the eye comprises obtaining the OCT image of the eye with one or more lenses achromatized for optical dispersion for the OCT light beam within a wavelength range of the OCT light source and for a field of view of the OCT image.

49. The method of claim 47, further comprising using a swept-source laser as the OCT light source.

50. The method of claim 47, further comprising presenting the OCT image on a display on the handheld imaging probe.

51. The method of claim 47, wherein illuminating the eye with an OCT light beam comprises illuminating the eye with an OCT light beam by coupling the OCT light beam from the OCT light source to the handheld imaging probe with a polarization maintaining optical fiber with a plurality of turns to remove the residual OCT light beam in one of axes of the polarization maintaining optical fiber.

52. A method of obtaining a wide field of view optical coherence tomography (OCT) image, the method comprising:
holding a handheld imaging probe in a hand,
placing an optical window disposed at a distal end of the imaging probe in contact with a cornea of an eye;
illuminating the eye with an OCT light beam from an OCT light source optically coupled to the handheld imaging probe,
scanning a first portion of the OCT light beam by using a scanning MEMS mirror; and
obtaining the OCT image through one or more lenses in an imaging path, the one or more lenses are achromatized for optical dispersion for the light beams within a wavelength range of the OCT light source and for a field of view of an OCT imaging module disposed in the handheld imaging probe to increase the OCT image quality.

53. The method of claim 52, further comprising using a swept-source laser as the OCT light source.

54. The method of claim 52, further comprising presenting the OCT image on a display on the handheld imaging probe.
